(19) United States
(12) Patent Application Publication
(10) Pub. No.: US 2026/0317811 A1
(43) Pub. Date: Oct. 15, 2026

(54) Detecting the Activity of RNA-Modulating Drugs Using ADAR Editing

(71) Applicant: The Board of Trustees of the Leland Stanford Junior University, Stanford, CA (US)

(72) Inventors: Xiaojing Gao, Stanford, CA (US); Natalie S. Kolber, Stanford, CA (US)

(21) Appl. No.: 19/485,065

(22) PCT Filed: May 29, 2024
(86) PCT No.: PCT/US2024/031504

§ 371 Date: Nov. 17, 2025

Related U.S. Application Data
(60) Provisional application No. 63/469,761, filed on May 30, 2023.
(60) Provisional application No. 63/642,429, filed on May 3, 2024.

(51) Int. Cl. C12Q 1/6825; C12Q 1/6811; C12Q 1/6827; C12Q 1/6876

(52) U.S. Cl. CPC C12Q 1/6825; C12Q 1/6811; C12Q 1/6827; C12Q 1/6876; C12Q 2600/156

(57) ABSTRACT

The present disclosure provides a method for screening a candidate agent for activity in altering a target RNA in a cell, the method including: a) contacting the candidate agent with the cell containing a sensor RNA, wherein the sensor RNA contains: (i) a first nucleotide sequence containing a sensor nucleotide sequence that hybridizes to the target RNA, wherein the sensor nucleotide sequence contains one or more editable codons, (ii) a second nucleotide sequence encoding a first cleavage domain, and (iii) a third nucleotide sequence encoding an output protein; and b) assaying for the presence of the output protein to assess the activity of the candidate agent to alter the target RNA.

### CROSS-REFERENCE TO RELATED APPLICATION

Pursuant to 35 U.S.C. § 119 (e), this application claims priority to the filing date of U.S. Provisional Application Ser. No. 63/469,761 filed on May 30, 2023 and U.S. Provisional Application Ser. No. 63/642,429 filed on May 3, 2024, the disclosures of which are herein incorporated by reference.

### GOVERNMENT RIGHTS

This invention was made with Government support under contract DGE-1656518 awarded by the National Science Foundation and under contracts EB027723, EB033858, and EB035891 awarded by the National Institutes of Health. The Government has certain rights in the invention.

### REFERENCE TO AN ELECTRONIC SEQUENCE LISTING

The contents of the electronic sequence listing (STAN-2197WO_Seq_List.xml; Size: 43,996 bytes; and Date of Creation: May 29, 2024) is herein incorporated by reference in its entirety.

### BACKGROUND

Drugs that act on the genetic level provide a compelling alternative to drugs that act on the protein level, particularly in diseases where there is a strong genetic component and/or where the protein of interest is considered undruggable. These include gene replacement therapies (where a faulty gene is replaced by a therapeutic gene by either DNA or RNA delivery); gene editing (where a toxic gene is knocked-down at the DNA level or a non-functional gene is edited into a functional gene using approaches including Cas9, zinc finger nucleases, base editing, or prime editing), RNA editing (where specific base edits are made to genes at the RNA level), small oligonucleotide therapies (where a gene is modulated at the RNA level by a variety of approaches including knockdown or inducing alternative splicing), and small molecules (that have been shown to act by a variety of mechanisms including regulation of splicing and translation inhibition). Examples of diseases that can or have been treated by these mechanisms include hemophilia A (gene therapy); sickle cell disease (gene editing); spinal muscular atrophy, SMA (modulation of splicing by either ASOs or small molecules); HCV infection (miRNA inhibition by ASOs), ATTR amyloidosis and Alzheimer's disease (siRNA knockdown). Notably, the approval in 2022 of Risdiplam, a small molecule that selectively modulates splicing for the treatment of SMA, suggests that there is broad potential to modulate RNA using small molecules via a variety of mechanisms, replacing more costly and difficult-to-administer oligonucleotide therapies.

However, methods for screening for such small molecules are lacking. Existing approaches for screening for small molecules that act on RNA may report only on binding (rather than the functional effects of the small molecule); be costly and time-consuming; and/or require knocking-in a splicing reporter cassette that may fail to recapitulate expression in the context of the native pre-mRNA. The need for inexpensive and fast readouts is especially critical when many drug candidates are screened in high-throughput and/or when developing a patient-specific therapy targeting a mutation that affects only one or a few patients.

### SUMMARY

The present disclosure provides a method for screening a candidate agent for activity in altering a target RNA in a cell, the method including: a) contacting the candidate agent with the cell containing a sensor RNA, wherein the sensor RNA contains: (i) a first nucleotide sequence containing a sensor nucleotide sequence that hybridizes to the target RNA, wherein the sensor nucleotide sequence contains one or more editable codons, (ii) a second nucleotide sequence encoding a first cleavage domain, and (iii) a third nucleotide sequence encoding an output protein; and b) assaying for the presence of the output protein to assess the activity of the candidate agent to alter the target RNA.

Kits for practicing the subject methods are also provided.

### BRIEF DESCRIPTION OF THE FIGURES

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. A schematic of an embodiment of the invention for detecting splice variants.

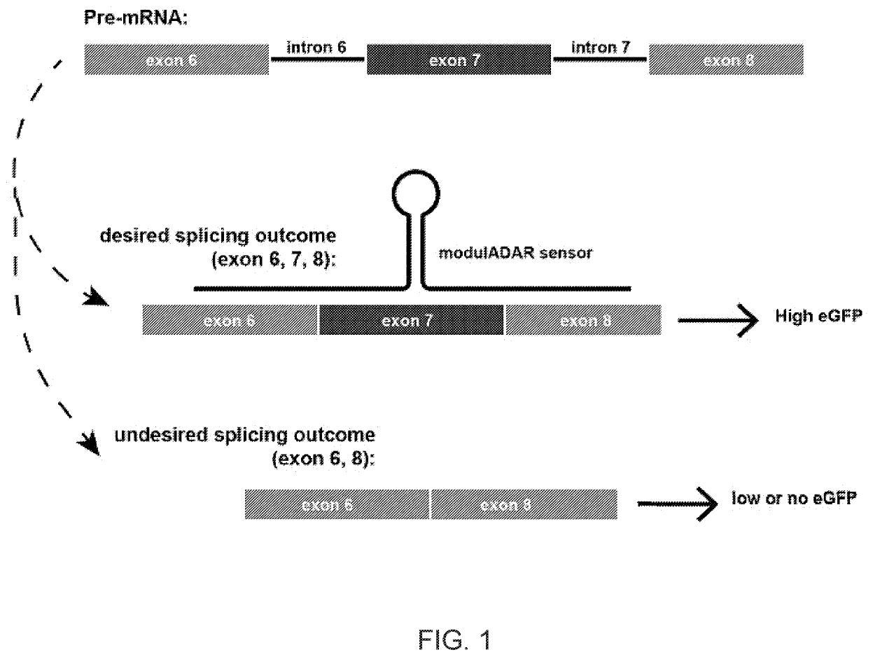

FIG. 2 depicts detection of alternatively spliced variants using sensor RNAs with varying sensor nucleotide lengths.

FIG. 3 depicts a comparison of the activity of modulADAR and linear sensors with varying editable codons in detecting splice variants of SMN2.

FIG. 4 depicts a schematic of an exemplary NOT sensor RNA.

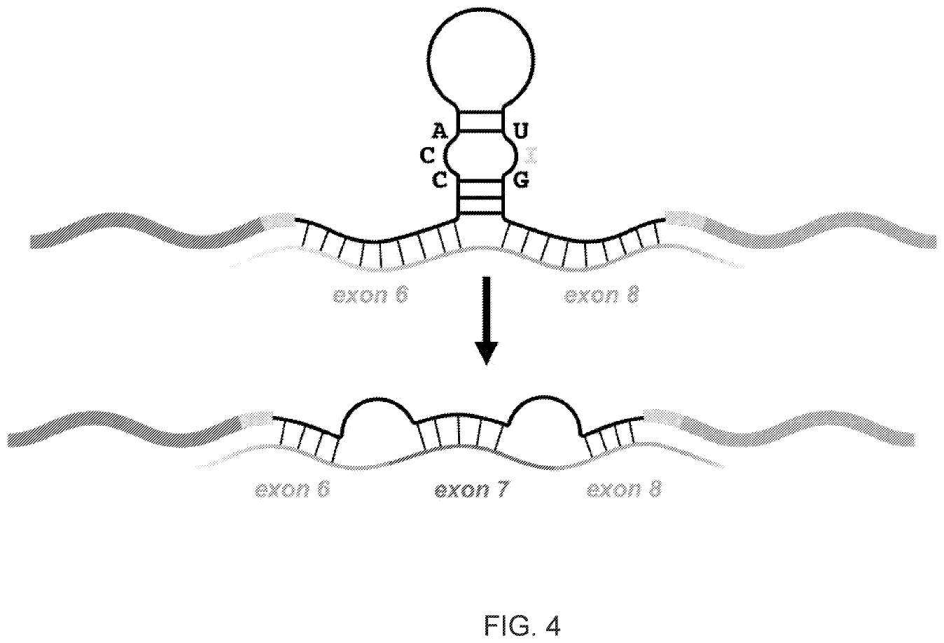

FIG. 5 depicts a comparison of the activity of NOT sensor RNAs having different sensor nucleotide lengths targeting SMN2.

FIG. 6 depicts a comparison of the activity of NOT sensor RNAs having different sensor nucleotide lengths targeting SCN1A.

FIG. 7 depicts a schematic of an exemplary NOT low background sensor RNA.

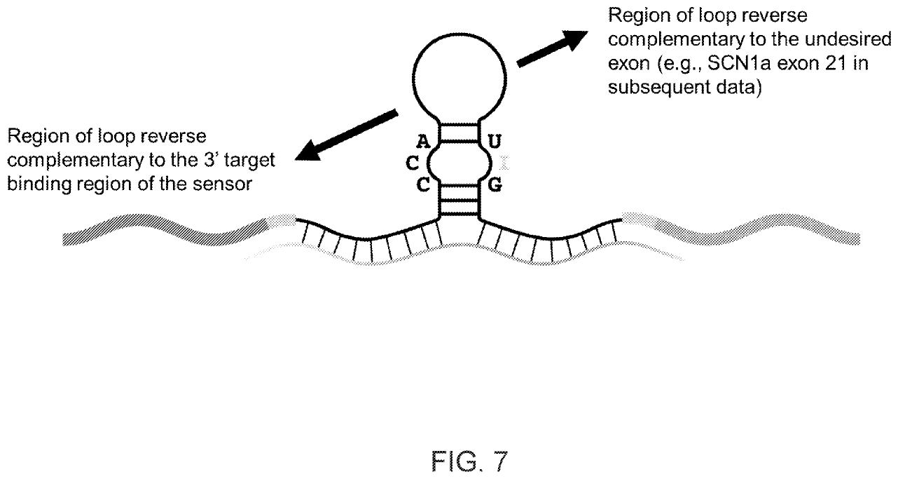

FIG. 8 depicts a comparison of exemplary low background sensor RNAs having differing loop lengths in the stem-loop sequences targeting SMN2 exon 7.

FIG. 9 depicts a comparison of exemplary low background sensor RNAs having differing loop lengths in the stem-loop sequences targeting SMN2 exon 7.

FIG. 10 depicts a comparison of exemplary low background NOT sensor RNAs having differing loop lengths in the stem-loop sequences targeting SMN2 exon 7.

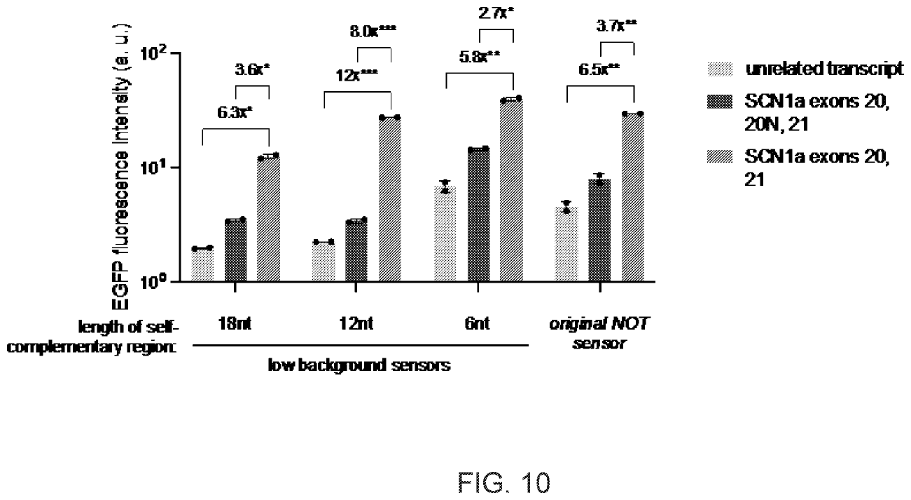

FIG. 11 depicts a schematic of SMN2 splicing.

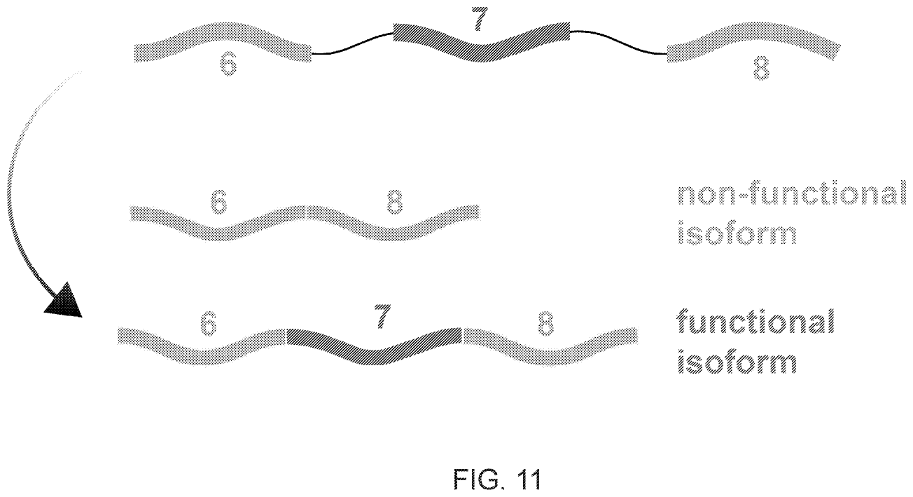

FIG. 12 depicts a schematic of SCN1A splicing.

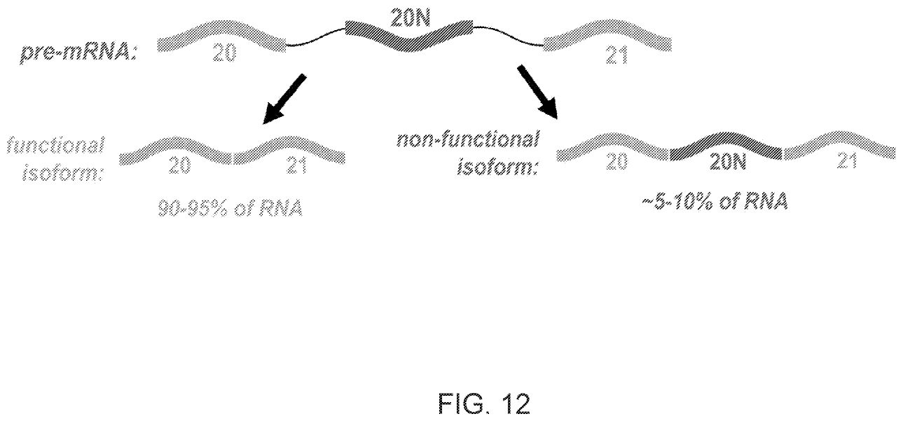

### DEFINITIONS

Before describing exemplary embodiments in greater detail, the following definitions are set forth to illustrate and define the meaning and scope of the terms used in the description.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It must be noted that as used herein and in the appended claims, the singular forms “a”, “an”, and “the” include plural referents unless the context clearly dictates otherwise. For example, the term “a RNA sensor” refers to one or more RNA sensors, i.e., a single RNA sensor and multiple RNA sensors. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

By “hybridizable” or “complementary” or “substantially complementary” it is meant that a nucleic acid (e.g. RNA, DNA) contains a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). Inosine (I) bases pair with cytosine/cytidine. In addition, for hybridization between two RNA molecules (e.g., dsRNA), and for hybridization of a DNA molecule with an RNA molecule (e.g., when a DNA target nucleic acid base pairs with a guide RNA, etc.): G can also base pair with U. For example, G/U base-pairing is partially responsible for the degeneracy (i.e., redundancy) of the genetic code in the context of tRNA anti-codon base-pairing with codons in mRNA. Thus, in the context of this disclosure, a G (e.g., of a protein-binding segment (e.g., dsRNA duplex) of a guide RNA molecule; of a target nucleic acid (e.g., target DNA or RNA) base pairing with a sensor RNA) is considered complementary to both a U and to C. For example, when a G/U base-pair can be made at a given nucleotide position of a protein-binding segment (e.g., dsRNA duplex) of a sensor RNA molecule, the position is not considered to be non-complementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Sensor nucleotide sequences that hybridize to target RNAs are reverse complementary or partially reverse complementary to the target RNA.

It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can include 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul ct al., J. Mol. Biol., 1990, 215, 403-410; Zhang and Madden, Genomc Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The term “naturally-occurring” as used herein as applied to a nucleic acid, a protein, a cell, or an organism, refers to a nucleic acid, protein, cell, or organism that is found in nature. For example, a polypeptide or polynucleotide sequence that is present in an organism (including viruses) that can be isolated from a source in nature and which has not been intentionally modified by a human in the laboratory is naturally occurring.

The term “exogenous” as used herein as applied to a nucleic acid or a protein refers to a nucleic acid or protein that is not normally or naturally found in and/or produced by a given bacterium, organism, or cell in nature. As used herein, the term “endogenous nucleic acid” refers to a nucleic acid that is normally found in and/or produced by a given bacterium, organism, or cell in nature. An “endogenous nucleic acid” is also referred to as a “native nucleic acid” or a nucleic acid that is “native” to a given bacterium, organism, or cell. As used herein, the term “endogenous polypeptide” refers to a polypeptide that is normally found in and/or produced by a given bacterium, organism, or cell in nature.

“Recombinant,” as used herein, means that a particular nucleic acid or protein is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA containing the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms.

Thus, e.g., the term “recombinant” nucleic acid or “recombinant” protein refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

By “construct” or “vector” is meant a recombinant nucleic acid, generally recombinant DNA, which has been generated for the purpose of the expression and/or propagation of a nucleotide sequence(s) of interest, or is to be used in the construction of other recombinant nucleotide sequences.

The term “transformation” or “transfection” refers to a permanent or transient genetic change induced in a cell following introduction of a nucleic acid (i.e., DNA and/or RNA exogenous to the cell). Genetic change (“modification”) can be accomplished either by incorporation of the new DNA into the genome of the host cell, or by transient or stable maintenance of the new DNA as an episomal element. Where the cell is a eukaryotic cell, a permanent genetic change is generally achieved by introduction of the DNA into the genome of the cell. Suitable methods of genetic modification include viral infection, transfection, conjugation, protoplast fusion, electroporation, particle gun technology, calcium phosphate precipitation, direct microinjection, transposon based, CRISPR/Cas, and the like. The choice of method is generally dependent on the type of cell being transformed and the circumstances under which the transformation is taking place (i.e. in vitro, ex vivo, or in vivo). A general discussion of these methods can be found in Ausubel et al, Short Protocols in Molecular Biology, 3rd ed., Wiley & Sons, 1995.

The terms “regulatory region” and “regulatory elements”, used interchangeably herein, refer to transcriptional and translational control sequences, such as promoters, enhancers, polyadenylation signals, terminators, protein degradation signals, translational start and stop codons, translation initiation sites, splice enhancer/donor/branch/acceptor sites, and the like, that provide for and/or regulate expression of a coding sequence and/or production of an encoded polypeptide in a host cell.

As used herein, a “promoter sequence” or “promoter” is a DNA regulatory region capable of binding/recruiting RNA polymerase (e.g., via a transcription initiation complex) and initiating transcription of a downstream (3′ direction) sequence (e.g., a protein coding (“coding”) or non protein-coding (“non-coding”) sequence. A promoter can be a constitutively active promoter (e.g., a promoter that is constitutively in an active/“ON” state), it may be an inducible promoter (e.g., a promoter whose state, active/“ON” or inactive/“OFF”, is controlled by an external stimulus, e.g., the presence of a particular temperature, compound, or protein), it may be a spatially restricted promoter (e.g., tissue specific promoter, cell type specific promoter, etc.), and/or it may be a temporally restricted promoter (e.g., the promoter is in the “ON” state or “OFF” state during specific stages of embryonic development or during specific stages of a biological process, e.g., hair follicle cycle in mice).

“Operably linked” refers to a juxtaposition wherein the components so described are in a relationship permitting them to function in their intended manner. For instance, a promoter is operably linked to a nucleotide sequence (e.g., a protein coding sequence, e.g., a sequence encoding an mRNA; a non protein coding sequence, e.g., a sequence encoding a Shh protein; and the like) if the promoter affects its transcription and/or expression.

The term “adenosine deaminase acting on RNA” or “ADAR” refers to an enzyme that catalyze the hydrolytic C6 deamination of adenosine (A) to produce inosine (I) in RNA substrates that are double stranded. ADARs preferentially edit double stranded RNAs at sites of mismatches where mismatches containing adenosines and cytosines are editing more efficiently than other mismatches. Editing by ADARs results in nucleotide substitution in RNA, because the purine I generated as the result of the deamination reaction is recognized as G instead of A, both by ribosomes during translational decoding of mRNA and by RNA-dependent polymerases during RNA replication. The term “ADAR” encompasses any know type of ADAR such as ADAR1 (ADAR) or ADAR2 (ADARB2).

As used herein “ADAR1” refers to an adenosine deaminase acting on RNA that catalyze the hydrolytic C6 deamination of adenosine (A) to produce inosine (I) in RNA substrates that are double stranded. ADAR1 has 2 main isoforms, p150 and p110. The term “ADAR1” encompasses ADAR1 from various species. Amino acid sequences of ADAR1 from various species are publicly available. See, e.g., GenBank Accession Nos. NP_001102 (Homo sapiens ADAR1 p150), NP_001180424.1 (Homo sapiens ADAR1 p110), NP_001139768 (Mus musculus ADAR1 p150), NP_001033676 (Mus musculus ADAR1 p110). The term “ADAR1” as used herein also encompasses fragments, fusion proteins, and variants (e.g., variants having one or more amino acid substitutions, addition, deletions, and/or insertions) that retain ADAR1 enzymatic activity.

As used herein “ADAR2” refers to an adenosine deaminase acting on RNA that catalyze the hydrolytic C6 deamination of adenosine (A) to produce inosine (I) in RNA substrates that are double stranded. ADAR2 is exclusively localized to the nucleus. The term “ADAR2” encompasses ADAR2 from various species. Amino acid sequences of ADAR2 from various species are publicly available. Scc, e.g., GenBank Accession Nos. NP_056648.1 (Homo sapiens ADAR2), NP_001020008.1 (Mus musculus ADAR2), ACO52474.1 (Doryteuthis opalescens ADAR2). The term “ADAR2” as used herein also encompasses fragments, fusion proteins, and variants (e.g., variants having one or more amino acid substitutions, addition, deletions, and/or insertions) that retain ADAR2 enzymatic activity.

The term “sample” as used herein relates to a material or mixture of materials, typically, although not necessarily, in fluid, i.e., aqueous, form, containing one or more components of interest. Samples may be derived from a variety of sources such as from food stuffs, environmental materials, a biological sample or solid, such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components). In certain embodiments of the method, the sample includes a cell. In some instances of the method, the cell is in vitro. In some instances of the method, the cell is in vivo.

The term “biological sample” encompasses a clinical sample or a non-clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's sample cell, e.g., a sample containing polynucleotides and/or polypeptides that is obtained from a patient's sample cell (e.g., a cell lysate or other cell extract containing polynucleotides and/or polypeptides); and a sample containing sample cells from a patient. A biological sample containing a sample cell from a patient can also include normal, non-diseased cells. A biological sample may be from a plant or an animal. The biological sample may also be from any species. In certain embodiments of the method, the biological sample includes a cell. In some instances of the method, the cell is in vitro. In some instances of the method, the cell is in vivo.

The term “editable codon” as used herein refers to a 3 nucleotide sequence that is editable by an ADAR protein or a derivative thereof. The codon may be a start codon, a stop codon or an AUA codon. The codon contains a sequence that contains an adenosine base. In general, in the methods disclosed herein, the editable codon is a start codon that is edited to become a non-start codon, a stop codon that is edited to become a non-stop codon, or a non-start codon (i.e. AUA) that is edited to become a start codon.

### DETAILED DESCRIPTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein arc for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

All patents and publications, including all sequences disclosed within such patents and publications, referred to herein are expressly incorporated by reference.

Where a range of values is provided, it is understood that each intervening value, to the tenth of the unit of the lower limit unless the context clearly dictates otherwise, between the upper and lower limit of that range and any other stated or intervening value in that stated range, is encompasscd within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

In further describing the subject invention, methods for screening a candidate agent for activity in altering a target RNA in a cell is described first in greater detail. Next, kits are described.

### METHODS FOR SCREENING A CANDIDATE AGENT FOR ACTIVITY IN ALTERING A TARGET RNA IN A CELL

As summarized above, methods are provided for screening a candidate agent for activity in altering a first target RNA in a cell, the method comprising: a) contacting the candidate agent with the cell comprising a sensor RNA, wherein the sensor RNA comprises: (i) a first nucleotide sequence comprising a sensor nucleotide sequence comprising: (a) a first region that is reverse complementary to the first target RNA and a second target RNA, (b) a second region comprising a stem-loop sequence comprising one or more editable codons wherein a first portion of the stem-loop sequence is reverse complementary to the second target RNA, and (c) a third region that is reverse complementary to the first target RNA and the second target RNA, and (ii) a second nucleotide sequence encoding a first cleavage domain, and (iii) a third nucleotide sequence encoding an output protein; and b) assaying for the presence of the output protein to assess the activity of the candidate agent to alter the target RNA, wherein the output protein is not produced when the first portion of the stem-loop sequence is hybridized to the second target RNA.

Candidate agents of the present disclosure may be any candidate agent that alters a target RNA in a cell. In some embodiments, the candidate agent is a nucleic acid. In some embodiments, the nucleic acid is an antisense oligonucleotide. In some embodiments, the nucleic acid is a small interfering RNA. In some embodiments, the candidate agent is a small molecule. In some embodiments, the candidate agent is a polypeptide. In some embodiments, the candidate agent is an antibody or fragment thereof. In some embodiments, the candidate agent is a gene-editing protein. In some embodiments, the gene editing protein is a CRISPR/Cas nuclease or variants thereof.

The methods are directed to a screening approach for identifying candidate agents that possess a desired activity. The desired activity is altering the target RNA. Altering the target RNA may result in a number of different outcomes including, without limitation, the target RNA is alternatively spliced, the target RNA is reduced in expression, the target RNA is increased in expression, the target RNA's nucleic acid sequence is changed, etc. In some embodiments, the reduction in the expression of the target RNA is a partial reduction in the expression of the target RNA. In some embodiments, the reduction in the expression of the target RNA is a complete loss of expression of the target RNA. In some embodiments, the increase in the expression of the target RNA is a partial increase in the expression of the target RNA. In some embodiments, the increase in the expression of the target RNA is an expression of the target RNA when the target RNA was not previously expressed.

The target RNA may be any RNA. For example, the target RNA includes, without limitation, mRNA, long non-coding RNA, transfer RNA, ribosomal RNA, small RNAs such as microRNA, small interfering RNA, small nucleolar RNAs, etc. In some embodiments, the target RNA may differentially expressed in different tissues cell types, or cell states. In some embodiments, the target RNA may be a genetic variant of gene. In these instances, the genetic variant may be predictive of a disease or susceptible to a disease such as an oncogenic mutation or a genetic variant associated with increased susceptibility to a pathogen. In some embodiments, the target RNA is associated with a disease or condition. In some embodiments, the methods of the present disclosure may be used to detect point mutations that are associated with a disease such as cancer, neurodegenerative disease, an autoimmune disease, etc. The target RNA may also be a gene fusion which may be associated with a cancer in general or a specific type of cancer. The target RNA may also be a specific splice variant (isoform) of a gene.

When the target RNA is a splice variant, there may be a first target RNA and a second target RNA. In some embodiments, the first target RNA is a non-pathogenic splice variant and the second RNA is a pathogenic splice variant. In some embodiments, the second target RNA is a non-pathogenic splice variant and the first RNA is a pathogenic splice variant. In some embodiments, the first and the second target RNAs are non-pathogenic variants. “Pathogenic splice variant” as used herein refers to a splice variant or a specific isoform of a gene as the result of alternative splicing that is associated with a disease or condition. The splice variants are of the same gene. The gene may be any gene that has a splice variant associated with a disease. Genes of interest include, without limitation, ABCA4, ABCB4, ABCD1, ACADSB, ADA, ADAMTS13, AGL, ALB, ALAS1, ALDH3A2, ALG6, APC, APOB, APP, AR, ATM, ATP7A, ATR, B2M, BCL6, BMP2K, BRCA1, BRCA2, BTK, C3, CAT, CD46, CDH1, CDH23, CFTR, CHM, COL11A1, COL11A2, COL1A1, COL1A2, COL2A1, COL3A1, COL4A5, COL6A1, COL6A3, COL7A1, COL9A2, COLQ, CUL4B, CYBB, CYP17, CYP19, CYP27, CYP27A1, DES, DMD, DYSF, EGFR, EMD, ESR1, ETV4, F13A1, F5, F7, F8, FAH, FANCA, FANCC, FANCG, FBN1, FECH, FGA, FGFR2, FGG, FIX, FLNA, FOXM1, FRAS1, GALC, GH1, GHV, HADHA, HAO1, HBA2, HBB, HEXA, HEXB, HLCS, HMBS, HMGCL, HNF1A, HPRT1, HPRT2, HSF4, HSPG2, HTT, IDS, IKBKAP, INSR, ITGB2, ITGB3, JAG1, KRAS, KRT5, KRAS, LICAM, LAMA3, LDLR, LMNA, LPL, LRRK2, MADD, MAPT, MeCP2, MLH1, MSH2, MSTIR, MTHFR, MUT, MVK, MYC, NF1, NF2, OAT, OPA1, OTC, PAH, PBGD, PCCA, PCSK9, PDH1, PGK1, PHEX, PKD2, PKLR, PLEKHM1, PLKR, POMT2, PRDM1, PRKAR1A, PROC, PSEN1, PTCHI, PTEN, PYGM, RP6KA3, RPGR, RSK2, SBCAD, SCN1A, SCN5A, SERPINA1, SERPINC1, SLC12A3, SLC26A4, SLC6A8, SMN2, SPINK5, SPTA1, SYNGAP1, TP53, TRAPPC2, TSC1, TSC2, TSHB, TTR, UGT1A1, and USH2A.

In some embodiments, the first target RNA is an RNA containing exons 6 and 8 of SMN2 and the second target RNA is an RNA containing exons 6, 7, and 8 of SMN2. In some embodiments, the first region is reverse complementary to exon 8 of SMN2, the third region is reverse complementary to exon 6 of SMN2, and the first portion of the stem-loop sequence is reverse complementary to exon 7 of SMN2. In some embodiments, the third region is reverse complementary to exon 8 of SMN2, the first region is reverse complementary to exon 6 of SMN2, and the first portion of the stem-loop sequence is reverse complementary to exon 7 of SMN2.

In some embodiments, the first target RNA is an RNA containing exons 20 and 21 of SCN1A and the second target RNA is an RNA containing exons 20, 20N, and 21 of SCN1A. In some embodiments, the first region is reverse complementary to exon 21 of SCN1A, the third region is reverse complementary to exon 20 of SCN1A, and the first portion of the stem-loop sequence is reverse complementary to exon 20N of SCN1A. In some embodiments, the third region is reverse complementary to exon 21 of SCN1A, the first region is reverse complementary to exon 20 of SCN1A, and the first portion of the stem-loop sequence is reverse complementary to exon 20N of SCN1A.

Duchenne muscular dystrophy is caused by mutations in dystrophin (DMD) which result in alternative splicing events and truncations of DMD. The most common mutations in DMD are located in exons 45-55. Work in the field has shown that skipping specific exons, e.g., exon 51, exon 53, and exon 45, may result in truncated variants of DMD that are still functional, such as disclosed by Eser et al. (Genes (Basel). 2022 July; 13 (7): 1241.) which is specifically incorporated by reference herein. In some embodiments, the first target RNA is an RNA containing exons 50 and 52 of DMD and the second target RNA is an RNA containing exons 50, 51, and 52 of DMD. In some embodiments, the first region is reverse complementary to exon 52 of DMD, the third region is reverse complementary to exon 50 of DMD, and the first portion of the stem-loop sequence is reverse complementary to exon 51 of DMD. In some embodiments, the first target RNA is an RNA containing exons 52 and 54 of DMD and the second target RNA is an RNA containing exons 52, 53, and 54 of DMD. In some embodiments, the first region is reverse complementary to exon 54 of DMD, the third region is reverse complementary to exon 52 of DMD, and the first portion of the stem-loop sequence is reverse complementary to exon 53 of DMD. In some embodiments, the first target RNA is an RNA containing exons 44 and 46 of DMD and the second target RNA is an RNA containing exons 44, 45, and 46 of DMD. In some embodiments, the first region is reverse complementary to exon 46 of DMD, the third region is reverse complementary to exon 44 of DMD, and the first portion of the stem-loop sequence is reverse complementary to exon 45 of DMD.

Laminopathies are a group of disorders associated with mutations in the Lamin (LMNA) gene. A number of missense mutations have been discovered in exons 4, 5, and 11 of the LMNA gene of individuals having Laminopathies. In some embodiments, the first target RNA is an RNA containing exons 3 and 5 of LMNA and the second target RNA is an RNA containing exons 3, 4, and 5 of LMNA. In some embodiments, the first region is reverse complementary to exon 5 of LMNA, the third region is reverse complementary to exon 3 of LMNA, and the first portion of the stem-loop sequence is reverse complementary to exon 4 of LMNA. In some embodiments, the first target RNA is an RNA containing cxons 4 and 6 of LMNA and the second target RNA is an RNA containing exons 4, 5, and 6 of LMNA. In some embodiments, the first region is reverse complementary to exon 6 of LMNA, the third region is reverse complementary to exon 4 of LMNA, and the first portion of the stem-loop sequence is reverse complementary to exon 5 of LMNA. In some embodiments, the first target RNA is an RNA containing exons 10 and 12 of LMNA and the second target RNA is an RNA containing exons 10, 11, and 12 of LMNA. In some embodiments, the first region is reverse complementary to exon 12 of LMNA, the third region is reverse complementary to exon 10 of LMNA, and the first portion of the stem-loop sequence is reverse complementary to exon 11 of LMNA.

Huntington's disease is caused by CAG repeat expansion in the HTT gene resulting in an expanded polyglutamine stretch at the N-terminal of the huntingtin protein. An important event in HD pathogenesis appears to be the proteolysis of the mutant protein, which forms N-terminal huntingtin fragments. Important cleavage sites are encoded by exon 12 of HTT. Work in the field has suggested exon 12 skipping as a potential therapeutic strategy, see, for example, Bhattacharyya et al. (Nat Commun. 2021 Dec. 15;12 (1): 7299) which is specifically incorporated by reference herein. In some embodiments, the first target RNA is an RNA containing exons 11 and 13 of HTT and the second target RNA is an RNA containing exons 11, 12, and 13 of HTT. In some embodiments, the first region is reverse complementary to exon 13 of HTT, the third region is reverse complementary to exon 11 of HTT, and the first portion of the stem-loop sequence is reverse complementary to exon 12 of HTT.

Pathogenic variation in SYNGAP1, the gene encoding SynGAP proteins, is a leading cause of sporadic neurodevelopmental disorders (NDDs) defined by impaired cognitive function, seizure, autistic features, and challenging behaviors. SYNGAP1 is completely intolerant of loss-of-function (LOF) variants. The last three exons of Syngap1 undergo alternative splicing which results in four distinct C-termini (a1, a2, b, g). Exon 19 is spliced into two reading frames (e19b/e19a). Because e19b lacks a stop codon, coding sequences from e20 and e21 are also included in mature transcripts. This leads to expression of a1, a2, or y C-terminal isoforms, and a1 and a2 arise from the absence of e20, but inclusion of e21. Work in the field has shown that exon skipping of exon 19a results in increased expression of the a1 and a2 which were shown to improve cognitive function and seizure protection. In some embodiments, the first target RNA is an RNA containing exons 18 and 19a of SYNGAP1 and the second target RNA is an RNA containing exons 18, 19b, and 21 of SYNGAP1. In some embodiments, the first region is reverse complementary to exon 18 of SYNGAP1, the third region is reverse complementary to exon 21 of SYNGAP1, and the first portion of the stem-loop sequence is reverse complementary to exon 19a of SYNGAP1.

Ataxia telangiectasia (AT) is a progressive autosomal recessive neurodegenerative disorder resulting from mutations in the ataxia telangiectasia mutated (ATM) gene. Approximately half of the mutations in AT have been reported as splicing mutations that either create new cryptic splice sites or interfere with splice regulatory elements. Some of these mutations elicit the activation of 5′ or 3′ cryptic splice sites within exonic regions, leading to the deletion of a part of the exon. In patients having AT, it has been shown that there can be mutations that result in cryptic splice sites in exon 55 and exon 8, see, for example, Siva et al. (Nucleic Acid Ther. 2014 Feb. 1; 24 (1): 69-86.) which is specifically incorporated by reference herein. In some embodiments, the first target RNA is an RNA containing exons 54 and 56 of ATM and the second target RNA is an RNA containing exons 54, 55, and 56 of ATM. In some embodiments, the first region is reverse complementary to exon 56 of ATM, the third region is reverse complementary to exon 54 of ATM, and the first portion of the stem-loop sequence is reverse complementary to exon 55 of ATM. In some embodiments, the first target RNA is an RNA containing exons 7 and 9 of ATM and the second target RNA is an RNA containing exons 7, 8, and 9 of ATM. In some embodiments, the first region is reverse complementary to exon 9 of ATM, the third region is reverse complementary to exon 7 of ATM, and the first portion of the stem-loop sequence is reverse complementary to exon 8 of ATM.

A wide spectrum of SLC26A4 mutations causes Pendred syndrome and enlarged vestibular aqueduct, both associated with sensorincural hearing loss (SNHL). A splice-site mutation, c.919-2A>G (A-2G) impairs the 3′ splice site of intron 7, resulting in exon 8 skipping during pre-mRNA splicing and a subsequent frameshift that creates a premature termination codon in the following exon, see, for example, Feng et al. (Mol Ther Nucleic Acids. 2022 Jun. 14; 28:280-292). In some embodiments, the first target RNA is an RNA containing exons 7 and 9 of SCL26A4 and the second target RNA is an RNA containing exons 7, 8, and 9 of SCL26A4. In some embodiments, the first region is reverse complementary to exon 9 of SCL26A4, the third region is reverse complementary to exon 7 of SCL26A4, and the first portion of the stem-loop sequence is reverse complementary to exon 8 of SCL26A4.

Familial hypercholesterolemia (FH) is a genetic disorder caused by variants in PCSK9 (proprotein convertase subtilisin/kexin type 9) gene. These genetic variants can cause elevation of low-density lipoprotein cholesterol (LDL-C) levels and tendon or skin xanthomas, which lead to a higher risk of atherosclerotic cardiovascular disease (ASCVD) in FH. Work in the field has shown that skipping exon 8 or 2 results in truncated forms of PCSK9 which has been a proposed treatment for FH, see, for example, Cale et al. (Sci Rep. 2023; 13:19725.). In some embodiments, the first target RNA is an RNA containing exons 7 and 9 of PCSK9 and the second target RNA is an RNA containing exons 7, 8, and 9 of PCSK9. In some embodiments, the first region is reverse complementary to exon 9 of PCSK9, the third region is reverse complementary to exon 7 of PCSK9, and the first portion of the stem-loop sequence is reverse complementary to exon 8 of PCSK9. In some embodiments, the first target RNA is an RNA containing exons 1 and 3 of PCSK9 and the second target RNA is an RNA containing exons 1, 2, and 3 of PCSK9. In some embodiments, the first region is reverse complementary to exon 3 of PCSK9, the third region is reverse complementary to exon 1 of PCSK9, and the first portion of the stem-loop sequence is reverse complementary to exon 2 of PCSK9.

Alterations in amyloid beta precursor protein (APP) have been implicated in cognitive decline in Alzheimer's disease (AD), which is accelerated in Down syndrome/Trisomy 21 (DS/TS21), likely due to the extra copy of the APP gene, located on chromosome 21. Proteolytic cleavage of APP generates amyloid-β (AB) peptide, the primary component of senile plaques associated with AD. Work in the field has proposed skipping exon 17 which contains proteolytic cleavage sites required for Aß peptide production, see, for example, Chang et al. (Mol Ther. 2018 Jun. 6; 26 (6): 1539-1551). In some embodiments, the first target RNA is an RNA containing exons 16 and 18 of APP and the second target RNA is an RNA containing cxons 16, 17, and 18 of APP. In some embodiments, the first region is reverse complementary to exon 18 of PCSK9, the third region is reverse complementary to exon 16 of APP, and the first portion of the stem-loop sequence is reverse complementary to exon 17 of APP.

Genetic and sporadic forms of Parkison's disease are associated with elevated leucine-rich repeat kinase 2 (LRRK2) activity. A reduction in activity of LRRK2 has been proposed as a treatment for Parkinson's disease. Work in the field has shown that skipping of exon 41 results in a reduction in activity of LRRK2, see, for example, Korecka et al. (Mol Ther Nucleic Acids. 2020 Sep. 4; 21:623-635) which is specifically incorporated by reference herein. In some embodiments, the first target RNA is an RNA containing exons 40 and 42 of LRRK2 and the second target RNA is an RNA containing exons 40, 41, and 42 of LRRK2. In some embodiments, the first region is reverse complementary to exon 42 of LRRK2, the third region is reverse complementary to cxon 40 of LRRK2, and the first portion of the stem-loop sequence is reverse complementary to exon 41 of LRRK2.

The androgen receptor (abbreviated AR) transcription factor is a major driver of prostate cancer pathology and is activated by androgen steroid hormones. Around 20 AR variant splice isoforms have been implicated in the development of hormone-refractory prostate cancer. The clinically most frequent AR splice variant, called ARv7, is produced by splicing inclusion of a cryptic exon called CE3 located within intron 3. ARv567es is a further cancer-associated AR splice form observed in patients and also encodes a constitutively active AR protein. ARv567es mRNA is made through skipping of exons 5-7 of the AR pre-mRNA and is only expressed within prostate cancer (and not the normal prostate), see, for example, Munkley et al. (Hum Genet. 2017; 136 (9): 1143-1154) which is specifically incorporated by reference herein. In some embodiments, the first target RNA is an RNA containing exons 3 and 4 of AR and the second target RNA is an RNA containing exons 3, CE3, and 4 of AR. In some embodiments, the first region is reverse complementary to exon 4 of AR, the third region is reverse complementary to exon 3 of AR, and the first portion of the stem-loop sequence is reverse complementary to exon CE3 of AR. In some embodiments, the first target RNA is an RNA containing exons 4 and 8 of AR and the second target RNA is an RNA containing exons 4, 6, and 8 of AR. In some embodiments, the first region is reverse complementary to exon 4 of PCSK9, the third region is reverse complementary to exon 8 of AR, and the first portion of the stem-loop sequence is reverse complementary to exon 6 of AR.

Collagen VI-related congenital muscular dystrophies (COL6-CMDs) are the second most common form of congenital muscular dystrophy. One of the most common mutations in COL6-CMD patients is a de novo deep intronic c.930+189C>T mutation in COL6A1 gene. This mutation creates a cryptic donor splice site and induces incorporation of a novel in-frame pseudo-exon in the mature transcripts. Work in the field has shown that exon skipping of the pseudoexon (PE) resulted in restoration of function COL6A1, see, for example, Aguti et al. (Mol Ther Nucleic Acids. 2020 Sep. 4; 21:205-216) which is specifically incorporated by reference herein. In some embodiments, the first target RNA is an RNA containing exons 11 and 12 of COL6A1 and the second target RNA is an RNA containing exons 12, PE, and 12 of COL6A1. In some embodiments, the first region is reverse complementary to exon 12 of COL6A1, the third region is reverse complementary to exon 11 of COL6A1, and the first portion of the stem-loop sequence is reverse complementary to exon PE of COL6A1.

The cells of the present disclosure may be any type of cell deemed useful for the screening methods disclosed herein. In some embodiments, the cell is a tissue-specific cell. In some embodiments, the cell is a disease-related cell. In some embodiments, the cell is a human patient-derived cell. When the cell is a human patient-derived cell, the patient may have a disease or condition. In some embodiments, the disease or condition is associated with a splice variant of a gene. Genes that have a splice variant that are associated with a disease or a condition may be any of the genes described above. The human patient-derived cell may then be used to screen for a candidate agent that treats the disease or condition. In some embodiments, the cell is present in an organoid. In some embodiments, the organoid is relevant to a disease or condition. In some embodiments, the organoid is derived from a human patient that has a disease or condition. In some embodiments, the cell is present in a non-human animal. In some embodiments, the non-human animal is a model for a disease or condition.

Cells of any origin are candidate cells for contacting the candidate agent of the present disclosure. Non-limiting examples of candidate cell types include connective tissue elements such as fibroblast, skeletal tissue (bone and cartilage), skeletal, cardiac and smooth muscle, epithelial tissues (e.g., liver, lung, breast, skin, bladder and kidney), neural cells (glia and neurons), endocrine cells (adrenal, pituitary, pancreatic islet cells), bone marrow cells, melanocytes, and many different types of hematopoetic cells. Suitable cells can also be cells representative of a specific body tissue from a subject. The types of body tissues include, but are not limited, to blood, muscle, nerve, brain, hcart, lung, liver, pancreas, spleen, thymus, esophagus, stomach, intestine, kidney, testis, ovary, hair, skin, bone, breast, uterus, bladder, spinal cord and various kinds of body fluids.

Cells suitable for use in a subject method include cells of a variety of subject hosts. Generally, such subject hosts are “mammals” or “mammalian”, where these terms are used broadly to describe organisms which are within the class mammalia, including the orders carnivore (e.g., dogs and cats), rodentia (e.g., mice, guinea pigs and rats), and primates (e.g., humans, chimpanzees and monkeys). In many aspects, the subject host will be a human. In certain embodiments, the subject host is a plant.

In some embodiments, the sensor RNA includes the following: (i) a first nucleotide sequence encoding a marker protein, (ii) a second nucleotide sequence encoding a first cleavage domain, (iii) a third nucleotide sequence including a sensor nucleotide sequence that hybridize to the target RNA, wherein the sensor nucleotide sequence includes one or more stop codons, (iv) a fourth nucleotide sequence encoding a second cleavage domain, and (v) a fifth nucleotide sequence encoding an output protein.

In some embodiments, the sensor RNA includes the following: (i) a first nucleotide sequence encoding a marker protein, (ii) a second nucleotide sequence encoding a cleavage domain (iii) a third nucleotide sequence including a sensor nucleotide sequence that hybridize to the 3′ UTR of the target RNA, wherein the sensor nucleotide sequence includes one or more stop codons, (iv) a fourth nucleotide sequence encoding a second cleavage domain, and (v) a fifth nucleotide sequence encoding an output protein.

In some embodiments, the sensor RNA includes the following: (i) a first nucleotide sequence containing a sensor nucleotide sequence that hybridizes to the target RNA wherein the sensor nucleotide sequence contains a stem-loop sequence containing one or more stop codons, (ii) a second nucleotide sequence encoding a cleavage domain, and (iii) a third nucleotide sequence encoding an output protein. In some embodiments, the first nucleotide sequence comprises (a) a first region that is reverse complementary to the first target RNA and a second target RNA, (b) a second region comprising a stem-loop sequence comprising one or more editable codons wherein a first portion of the stem-loop sequence is reverse complementary to the second target RNA, and (c) a third region that is reverse complementary to the first target RNA and the second target RNA.

In some embodiments, the sensor RNA includes the following: (i) a first nucleotide sequence encoding a marker protein, (ii) a second nucleotide sequence encoding a cleavage domain (iii) a third nucleotide sequence including a sensor nucleotide sequence that hybridizes to the target RNA wherein the sensor nucleotide sequence contains a stem-loop sequence containing one or more stop codons, (iv) a fourth nucleotide sequence encoding a second cleavage domain, and (v) a fifth nucleotide sequence encoding an output protein. In some embodiments, the third nucleotide sequence comprises (a) a first region that is reverse complementary to the first target RNA and a second target RNA, (b) a second region comprising a stem-loop sequence comprising one or more editable codons wherein a first portion of the stem-loop sequence is reverse complementary to the second target RNA, and (c) a third region that is reverse complementary to the first target RNA and the second target RNA.

In some embodiments, the sensor RNA includes the following: (i) a first nucleotide sequence comprising a sensor nucleotide sequence comprising: (a) a first region that is reverse complementary to the first target RNA and a second target RNA, (b) a second region comprising a stem-loop sequence comprising one or more editable codons wherein a first portion of the stem-loop sequence is reverse complementary to the second target RNA, and (c) a third region that is reverse complementary to the first target RNA and the second target RNA, and (ii) a second nucleotide sequence encoding a first cleavage domain, and (iii) a third nucleotide sequence encoding an output protein

In some embodiments, the sensor RNA has one or more stop codons containing one or more base that is mismatched with 1) a sequence within the stem-loop opposite the stop codon or 2) a sequence in the target RNA. The one or more bases that are mismatched are generally not more than 2 bases that are mismatched. In an embodiment, the sensor RNA has one or more stop codons containing only 1 base that is mismatched with 1) a sequence within the stem-loop opposite the stop codon or 2) a sequence in the target RNA. In some embodiments, the sensor RNA does not have any mismatched bases.

In certain embodiments, the sensor RNA contains the first nucleotide sequence to the third, fourth or fifth nucleotide sequences in order (i.e., the fifth nucleotide sequence follows the fourth nucleotide sequence which follows the third nucleotide sequence which follows the second nucleotide sequence which follows the first nucleotide sequence). In some embodiments, the sensor RNA contains the first nucleotide sequence to the third, fourth or fifth nucleotide sequence that are not in order described above.

In some embodiments, the sensor RNAs contain a 5′ RNA cap that is 5′ to the first nucleotide sequence. In some embodiments, the sensor RNA contains a sequence encoding a 5′ UTR that is 5′ to the first nucleotide sequence. In some embodiments, the sensor RNA contains a sequence encoding a 5′ UTR that is 5′ to the first nucleotide sequence and a 5′ RNA cap that is 5′ of the sequence encoding the 5′ UTR. In some embodiments, the sensor RNA contains a sequence encoding a 3′ UTR that is 3′ of the sequence encoding the output protein. In some embodiments, the sensor RNA contains a sequence encoding a polyA tail that is 3′ of the sequence encoding the output protein. In some embodiments, the sensor RNA contains a sequencing encoding a 3′ UTR that is 3′ of the sequence encoding the output protein and a sequence encoding a polyA tail that is 3′ of the sequence encoding the polyA tail. In some embodiments, the sensor RNA contains a sequence encoding a 5′ UTR that is 5′ to the first nucleotide sequence and a 5′ RNA cap that is 5′ of the sequence encoding the 5′ UTR, a sequencing encoding a 3′ UTR that is 3′ of the sequence encoding the output protein and a sequence encoding a polyA tail that is 3′ of the sequence encoding the output.

A 5′ cap can be a native 7-methylguanylate cap, or a cap analog, for example anti-reverse cap analog (ARCA), 3′-O-Me-m7G (5′) ppp (5′) G, (m7G (5′) ppp (5′) G), Cap0, Cap1, inosine, N1-methyl-guanosinc, 2′ fluoro-guanosinc, 7-dcaza-guanosine, 8-oxo-guanosinc, 2-amino-guanosine, LNA-guanosine, 2-azido-guanosine, etc.

The sensor RNA of the present disclosure contains a sensor nucleotide sequence or a stem-loop sequence containing one or more stop codons which is followed by a nucleotide sequence encoding an output protein. In some embodiments, the sensor RNA contains one or more stop codons that contain one or more base that is mismatched with the target RNA or the sequence within the stem-loop. In some embodiments, the sensor RNA does not contain any mismatches with the target RNA. In the presence of the target RNA, the sensor nucleotide sequence of the sensor RNA hybridizes to the target RNA thereby forming a double-stranded RNA molecule that can recruit an ADAR protein. The double-stranded RNA can contain a stop codon with or without mismatches, or a stop codon could be within the stem-loop of the sensor RNA. An ADAR protein then edits the adenosine base within the stop codon(s) of the sensor RNA to an inosine base. This editing removes the stop codon(s) which then allows the output protein to be produced from the sensor RNA within the biological sample.

When the sensor RNA contains a nucleotide sequence containing a stem-loop sequence comprising a stop codon, any stem-loop sequence may be used. In some embodiments, the stem-loop contains natural editing sites. Natural editing sites are sites within nucleotide sequences that are edited in nature. Natural editing sites are known in the art and have been described in, for example, Gabay et al. (Nat Commun. 2022 Mar. 4;13 (1): 1184) which is specifically incorporated by reference herein. Examples of natural editing sites include, without limitation, editing sites found in GRIA2, GRIA3, IGFBP7, NEIL1, FLNA, GRIK2, CDK13, GABRA3, GLI1, SPEG, HTR2C, GRIA4, CYFIP2, CADPS, CADPS, RICTOR, COG3, GRIK1, COPA, HBE1, SON, FLNB, MAGEL2, NOVA1, PNMT, WASH1, LAT, DACT3, FXYD5, ZNF717, ZNF551 CAPS1, etc. In some embodiments, the stem-loop sequence is a GluR-B stem-loop or a modified variant thereof. In some embodiments, the stem contains a natural editing site while the loop is a synthetic sequence. In some embodiments, the sequence of the stem is altered compared to the natural editing site by the addition or removal of nucleotides in order to add or remove mismatches. In some embodiments, the sequence alteration adds or removes additional stop codons In some embodiments, the stem-loop sequence contains a CAPS1 derived stem-loop according to:

(SEQ ID NO: 1)
CAAGGUCAAUGAGGAGAUGUACAUAGAAAUACAAUCCU
GUGUACAUCUUCUAGCAUGACCCAC.

In some embodiments, the stem-loop sequence contains a CAPS1 derived stem-loop according to:

(SEQ ID NO: 2)
CAAGGUCAAUGAGGAGAUGUACAUAAUACAAUGUGUACAU
CUUCUAGCAUGACCCAC.

In some embodiments, the stem-loop sequence contains a GLI1 derived stem-loop according to:

(SEQ ID NO: 3)
CCCAACCUCUGUCUACUCACCACAGCCCCCCAGCAUCACU
GUGAAUGCUGCCAUGGAUGCUAGAGGGCUACAGGAAGAGC
CAGAAGUUGG.

In some embodiments, the stem-loop sequence contains a GLI1 derived stem-loop according to:

(SEQ ID NO: 4)
CUCACCACAGCCCCCCAGCAUCACUGUGAAUGCUGCCAUGG
AUGCUAGAGGGCUACAGGA.

In some embodiments, the stem-loop sequence contains a GABRA3 derived stem-loop according to:

(SEQ ID NO: 5)
AAGUGGCAUAUGCGACGGCCAUGGACUGGUUCAUAGCCGU
CUGUUAUGCCU.

In some embodiments, the stem-loop sequence contains a GABRA3 derived stem-loop according to:

(SEQ ID NO: 6)
UGGCAUAUGCGACGGCCAUGGACUGGUUCAUAGCCGUCU
GUUAUG.

In some embodiments, the stem-loop sequence contains a GLURB derived stem-loop according to:

(SEQ ID NO: 7)
CAUUAAGGUGGGUGGAAUAGUAUACAAAGUAUCCCACCU
ACCCUGAUG.

In some embodiments, the stem-loop sequence contains a GLURB derived stem-loop according to:

(SEQ ID NO: 8)
CAUUAAGGUGGGUGGAAUAGUAUACAAAGUAUCCCACC
UACCCCGAUG.

In some embodiments, the stem-loop sequence comprises GLURB derived stem-loop according to:

(SEQ ID NO: 9)
UCCGUUUAGGUGGGUGGAAUAGUAAUACAAAGUAUCCC
ACCUACCCAGACG.

In some embodiments, the stem-loop sequence is according to:

(SEQ ID NO: 10)
CAUUUAGGUGGGUGGGCUAACCACCUACCCAGAUG.

In some embodiments, the stem-loop sequence is according to:

(SEQ ID NO: 11)
CAUUUAGGUGGGUGGAAUGCUAAAUCCCACCUACCCAGAUG.

In some embodiments, the stem-loop sequence is according to:

(SEQ ID NO: 12)
AUGCGACGGCCAUGGACUGGUUCAUAGCCGUCUGUU.

When the first target RNA is an RNA comprising exons 6 and 8 of SMN2, the sensor nucleotides sequence may have a specific nucleic acid sequence. In some embodiments, the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 13)
ATGGGATCCATAATTCCCCCACCACCTCCCATATGTCCAGATTCTCTTG
ATGATGCTGATGCTTTGGGAAGTATGTTAATTTCATGGTACATGAGTGG
CTATCATACTGGCTATTATATGGAAATGCTGGCATAGAGCAGCACGTCT
TCACACTCGAAGATTTCGTTGGGGACTGGCGACAGACAGCCGGCTACAA
CCTGGACCAAGTCCTTGAACAGGGAGGTGTGTCCAGTTTGTTTCAGAAT
CTCGGGGTGTCCGTAACTCCGATCCAAAGGATTGTCCTGAGCGGTGAAA
ATGGGCTGAAGATCGACATCCATGTCATCATCCCGTATGAAGGTCTGAG
CGGCGACCAAATGGGCCAGATCGAAAAAATTTTTAAGGTGGTGTACCCT
GTGGATGATCATCACTTTAAGGTGATCCTGCACTATGGCACACTGGTAA
TCGACGGGGTTACGCCGAACATGATCGACTATTTCGGACGGCCGTATGA
AGGCATCGCCGTGTTCGACGGCAAAAAGATCACTGTAACAGGGACCCTG
TGGAACGGCAACAAAATTATCGACGAGCGCCTGATCAACCCCGACGGCT
CCCTGCTGTTCCGAGTAACCATCAACGGAGTGACCGGCTGGCGGCTGTG
CGAACGCATTCTGGCGTAG.

When the second target RNA is an RNA comprising exons 6, 7, and 8 of SMN2, the sensor nucleotides sequence may have a specific nucleic acid sequence. In some embodiments, the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 14)
ATGGGATCCATAATTCCCCCACCACCTCCCATATGTCCAGATTCTCTTG
ATGATGCTGATGCTTTGGGAAGTATGTTAATTTCATGGTACATGAGTGG
CTATCATACTGGCTATTATATGGGTTTTAGACAAAATCAAAAAGAAGGA
AGGTGCTCACATTCCTTAAATATAAGGAGAAATGCTGGCATAGAGCAGC
ACGTCTTCACACTCGAAGATTTCGTTGGGGACTGGCGACAGACAGCCGG
CTACAACCTGGACCAAGTCCTTGAACAGGGAGGTGTGTCCAGTTTGTTT
CAGAATCTCGGGGTGTCCGTAACTCCGATCCAAAGGATTGTCCTGAGCG
GTGAAAATGGGCTGAAGATCGACATCCATGTCATCATCCCGTATGAAGG
TCTGAGCGGCGACCAAATGGGCCAGATCGAAAAAATTTTTAAGGTGGTG
TACCCTGTGGATGATCATCACTTTAAGGTGATCCTGCACTATGGCACAC
TGGTAATCGACGGGGTTACGCCGAACATGATCGACTATTTCGGACGGCC
GTATGAAGGCATCGCCGTGTTCGACGGCAAAAAGATCACTGTAACAGGG
ACCCTGTGGAACGGCAACAAAATTATCGACGAGCGCCTGATCAACCCCG
ACGGCTCCCTGCTGTTCCGAGTAACCATCAACGGAGTGACCGGCTGGCG
GCTGTGCGAACGCATTCTGGCGTAG.

When the first target RNA is an RNA comprising exons 20 and 21 of SCN1A, the sensor nucleotides sequence may have a specific nucleic acid sequence. In some embodiments, the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 23)
atgGTTTCATTGGTCAGTTTAACAGCAAATGCCTTGGGTTACTCAGAAC
TTGGAGCCATCAAATCTCTCAGGACACTAAGAGCTCTGAGACCTCTAAG
AGCCTTATCTCGATTTGAAGGGATGAGGGTGGTTGTGAATGCCCTTTTA
GGAGCAATTCCATCCATCATGAATGTGCTTCTGGTTTGTCTTATATTCT
GGCTAATTTTCAGCATCATGGGCGTAAATTTGTTTGCTGGCAAATTCTA
CCACTGTATTAACACCACAACTGGTGACAGGTTTGACATCGAAGACGTG
AATAATCATACTGATTGCCTAAAACTAATAGAAAGAAATGAGACTGCTC
GATGGAAAAATGTGAAAGTAAACTTTGATAATGTAGGATTTGGGTATCT
CTCTTTGCTTCAAGTTTGA.

When the second target RNA is an RNA comprising exons 20, 20N, and 21 of SCN1A, the sensor nucleotides sequence may have a specific nucleic acid sequence. In some embodiments, the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 22)
ATGGTTTCATTGGTCAGTTTAACAGCAAATGCCTTGGGTTACTCAGAAC
TTGGAGCCATCAAATCTCTCAGGACACTAAGAGCTCTGAGACCTCTAAG
AGCCTTATCTCGATTTGAAGGGATGAGGGATAATCTTGCTCCAACTTGG
ATGGGGTGGAGCGCTGGTTCCTCCCCTGAGCCCTTTATTATGGGTGGTT
GTGAATGCCCTTTTAGGAGCAATTCCATCCATCATGAATGTGCTTCTGG
TTTGTCTTATATTCTGGCTAATTTTCAGCATCATGGGCGTAAATTTGTT
TGCTGGCAAATTCTACCACTGTATTAACACCACAACTGGTGACAGGTTT
GACATCGAAGACGTGAATAATCATACTGATTGCCTAAAACTAATAGAAA
GAAATGAGACTGCTCGATGGAAAAATGTGAAAGTAAACTTTGATAATGT
AGGATTTGGGTATCTCTCTTTGCTTCAAGTTTGTGA.

In some embodiments, when sensor RNA is directed to SMN2 the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 20)
CGAAATCTTCGAGTGTGGAGACGTGCTGCTCTATGCCAGCATTTCTGGC
ATATGCGACGGCCATGGATCCTTATATTTAAGGAATGTGAGCACCTTCC
TTCTTTTTGGTTTTGTCTAAAACCTTCATAGCCGTCTGTTATGCATATA
ATAGCCAGTATGATAGCCACTCATGTACCATGGAATTCAA.

In some embodiments, when sensor RNA is directed to SMN2 the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 21)
CGAGTGTGGAGACGTGCTGCTCTATGCCAGCATTTCTGGCATATGCGAC
GGCCATGGATCCTTATATTTAAGGAATGTGAGCACCTTCCTTCTTTTTG
GTTTTGTCTAAAACCTTCATAGCCGTCTGTTATGCATATAATAGCCAGT
ATGATAGCCACTCATGTACCA.

In some embodiments, when sensor RNA is directed to SMN2 the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 27)
AGCATTTCTCCTTATATTGAAGGAATGCGAGCACCTTGGCATATGCGAC
GGCCATGGAAGGTGCTCGCATTTTCATAGCCGTCTGTTATGTCCTTCTT
TTTGATTTTGTCTAAAACCCATATAATA.

In some embodiments, when sensor RNA is directed to SMN2 the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 28)
AGCATTTCTCCTTATATTGAAGGAATGCGAGCACCTTGGCATATGCGAC
GGCCATGGAAGGTGCTCGCTTCATAGCCGTCTGTTATGTCCTTCTTTTT
GATTTTGTCTAAAACCCATATAATA.

In some embodiments, when sensor RNA is directed to SMN2 the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 29)
AGCATTTCTCCTTATATTGAAGGAATGCGAGCACCTTGGCATATGCGAC
GGCCATGGAAGGTGCTTTCATAGCCGTCTGTTATGTCCTTCTTTTTGAT
TTTGTCTAAAACCCATATAATA.

In some embodiments, when sensor RNA is directed to SMN2 the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 30)
AGCATTTCTCCTTATATTGAAGGAATGCGAGCACCTTGGCATATGCGAC
GGCCATGGAAGGTGCTCGCATTCCTTTCATAGCCGTCTGTTATGTCCTT
CTTTTTGATTTTGTCTAAAACCCATATAATA.

In some embodiments, when sensor RNA is directed to SMN2 the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 31)
AGCATTTCTCCTTATATTGAAGGAATGCGAGCACCTTGGCATATGCGAC
GGCCATGGAAGGTGCTCGCATTCCTTCATTCATAGCCGTCTGTTATGTC
CTTCTTTTTGATTTTGTCTAAAACCCATATAATA.

In some embodiments, when sensor RNA is directed to SMN2 the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 32)
AGCATTTCTCCTTATATTGAAGGAATGCGAGCACCTTGGCATATGCGAC
GGCCATGGAAGGTGCTCGCATTCCTTCAATATTCATAGCCGTCTGTTAT
GTCCTTCTTTTTGATTTTGTCTAAAACCCATATAATA.

In some embodiments, when sensor RNA is directed to SMN2 the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 40)
AGCAUUUCUCCUUAUAUUGAAGGAAUGCGAGCACCUUGGCAUAUGCGA
CGGCCAUGGACUGGUUCAUAGCCGUCUGUUAUGUCCUUCUUUUUGAUU
UUGUCUAAAACCCAUAUAAUA.

In some embodiments, when sensor RNA is directed to SMN2 the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 41)
CUCUAUGCCAGCAUUUCUCCUUAUAUUGAAGGAAUGCGAGCACCUUGG
CAUAUGCGACGGCCAUGGACUGGUUCAUAGCCGUCUGUUAUGUCCUUC
UUUUUGAUUUUGUCUAAAACCCAUAUAAUAGCCAGUAUG.

In some embodiments, when sensor RNA is directed to SMN2 the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 42)
GUGCUGCUCUAUGCCAGCAUUUCUCCUUAUAUUGAAGGAAUGCGAGCA
CCUUGGCAUAUGCGACGGCCAUGGACUGGUUCAUAGCCGUCUGUUAUG
UCCUUCUUUUUGAUUUUGUCUAAAACCCAUAUAAUAGCCAGUAUGAUA
GCC.

In some embodiments, when sensor RNA is directed to SCN1A the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 24)
AAGCACATTCATGATGGATGGAATTGCTCCTcAAAGGGCATTCACAACC
ACTGGCATATGCGACGGCCATGGACCATAATAAAGGGCTCAGGGGAGGA
ACCAGCGCTCCACCCCATCCAAGTTGGAGCAAGATTATCTTCATAGCCG
TCTGTTATGCCTCATCCCTTCAAATCGAGATcAGGCTCTTgGAGGTCTC
AGAGCTCTTgG.

In some embodiments, when sensor RNA is directed to SCN1A the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 25)
ATTCATGATGGATGGAATTGCTCCTcAAAGGGCATTCACAACCACTGGC
ATATGCGACGGCCATGGACCATAATAAAGGGCTCAGGGGAGGAACCAGC
GCTCCACCCCATCCAAGTTGGAGCAAGATTATCTTCATAGCCGTCTGTT
ATGCCTCATCCCTTCAAATCGAGATcAGGCTCTTgGAGGTCTCAGAGC.

In some embodiments, when sensor RNA is directed to SCN1A the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 26)
GGATGGAATTGCTCCTcAAAGGGCATTCACAACCACTGGCATATGCGAC
GGCCATGGACCATAATAAAGGGCTCAGGGGAGGAACCAGCGCTCCACCC
CATCCAAGTTGGAGCAAGATTATCTTCATAGCCGTCTGTTATGCCTCAT
CCCTTCAAATCGAGATcAGGCTCTTgGAGG.

In some embodiments, when sensor RNA is directed to SCN1A the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 33)
ATTCATGATGGATGGAATTGCTCCTcAAAGGGCATTCACAACCACTGGC
ATATGCGACGGCCATGGAGTGGTTGTGAATGCCCTTCCATAATAAAGGG
CTCAGGGGAGGAACCAGCGCTCCACCCCATCCAAGTTGGAGCAAGATTA
TCTTCATAGCCGTCTGTTATGCCTCATCCCTTCAAATCGAGATcAGGCT
CTTgGAGGTCTCAGAGC.

In some embodiments, when sensor RNA is directed to SCN1A the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 34)
ATTCATGATGGATGGAATTGCTCCTcAAAGGGCATTCACAACCACTGGC
ATATGCGACGGCCATGGAGTGGTTGTGAATCCATAATAAAGGGCTCAGG
CCCATCCAAGTTGGAGCAAGATTATCTTCATAGCCGTCTGTTATGCCTC
AGGAGGAACCAGCGCTCCACTCCCTTCAAATCGAGATcAGGCTCTTgGA
GGTCTCAGAGC.

In some embodiments, when sensor RNA is directed to SCN1A the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 35)
ATTCATGATGGATGGAATTGCTCCTcAAAGGGCATTCACAACCACTGGC
ATATGCGACGGCCATGGAGTGGTTCCATAATAAAGGGCTCAGGGGAGGA
ACCAGCGCTCCACCCCATCCAAGTTGGAGCAAGATTATCTTCATAGCCG
TCTGTTATGCCTCATCCCTTCAAATCGAGATcAGGCTCTTgGAGGTCTC
AGAGC.

In some embodiments, when sensor RNA is directed to SCN1A the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 36)
ATTCATGATGGATGGAATTGCTCCTcAAAGGGCATTCACAACCACTGGC
ATATGCGACGGCCATGGAGTGGTTGTGAATGCCCCATAATAAAGGGCTC
AGGGGAGGAACCAGCGCTCCACCCCATCCAAGTTGGAGCAAGATTATCT
TCATAGCCGTCTGTTATGCCTCATCCCTTCAAATCGAGATcAGGCTCTT
gGAGGTCTCAGAGC.

In some embodiments, when sensor RNA is directed to SCN1A the sensor nucleotide sequence contains a nucleotide sequence having at least 75%, at least 80%, at least 85%, at least 90%, at least 95%, at least 98%, at least 99%, or 100%, nucleotide sequence identity to the following nucleotide sequence:

(SEQ ID NO: 37)
ATTCATGATGGATGGAATTGCTCCTcAAAGGGCATTCACAACCACTGGC
ATATGCGACGGCCATGGAGTGGTTGTGCCATAATAAAGGGCTCAGGGGA
GGAACCAGCGCTCCACCCCATCCAAGTTGGAGCAAGATTATCTTCATAG
CCGTCTGTTATGCCTCATCCCTTCAAATCGAGATcAGGCTCTTgGAGGT
CTCAGAGC.

In the above embodiment, the stem-loop sequence has a first portion that is reverse complementary to the second target RNA. The first portion of the stem-loop sequence hybridizing to the second target RNA prevents the production of the output protein and thus the output protein is only produced when the sensor RNA is hybridized to the first target RNA. The stem-loop sequence has a stem sequence and a loop sequence. The stem sequence is the sequence of the stem-loop sequence that hybridizes with itself to form the stem of the stem-loop and the loop sequence is the sequence of the stem-loop sequence that does not hybridize with a sequence within the stem-loop sequence. In some embodiments, the first portion of the stem-loop sequence is contained within the loop sequence of the stem-loop sequence. Sensor RNAs containing a stem-loop sequence containing the first portion of the stem-loop sequence that is reverse complementary to the second target RNA may be referred to as a NOT sensor RNA. When a sensor RNA contains a stem-loop containing a first portion that is reverse complementary to the first region or the third region it may be referred to as a low background sensor RNA. The first portion of the stem-loop sequence may be any portion of the stem-loop sequence. In some embodiments, the first portion of the stem-loop sequence is contained in the loop of the stem-loop sequence. For instance, the first portion may be 1/10, 2/10, 3/10, 4/10, 5/10 or 1/2, 6/10, 7/10, 8/10, 9/10, 10/10, 1/4, or 3/4 of the stem-loop sequence or the loop sequence of the stem-loop sequence. The first portion may be 3 nucleotides, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 nucleotides, or more than 80 nucleotides. The first portion may be a range of nucleotides including, without limitation, 3-5 nucleotides, 5-10, 10-15, 15-20, 20-25, 25-30, 30-35, 35-40, 40-45, 45-50, 50-55, 55-60, 60-65, 65-70, 70-75, 75-80, 80-85, 10-20, 20-30, 30-40, 40-50, 50-60, 60-70, 70-80, etc.

In some embodiments, the sensor nucleotide sequence contains a first region that is reverse complementary to the target RNA, a second region containing a stem-loop sequence containing one or more editable codons, and a third region that is reverse complementary to the target RNA. By “first region”, “second region”, and “third region” it is meant a specific sequence of nucleotides in the sensor nucleotide sequence. In some embodiments, the first region is 5′ of the second region and the second region is 5′ of the third region. In some embodiments, the sensor nucleotide sequence contains a fourth region that is reverse complementary to the target RNA. In some embodiments, the sensor nucleotide region contains a sixth region that is reverse complementary to the target RNA. In some embodiments, the sensor nucleotide region contains a seventh region that is reverse complementary to the target RNA. In some embodiments, the sensor nucleotide region contains an eighth region that is reverse complementary to the target RNA. In some embodiments, the sensor nucleotide region contains a ninth region that is reverse complementary to the target RNA. In some embodiments, the sensor nucleotide region contains a tenth region that is reverse complementary to the target RNA. When the sensor nucleotide sequence has three or more regions that are reverse complementary to the target RNA, i.e., the first region, the third region, and the fourth region, the regions may be reverse complementary to discontinuous regions of the target RNA. In some embodiments, the discontinuous regions are the target RNA are different exons of a gene or gene product. Discontinuous regions of the target RNA may be regions of the target RNA that are separated by 10 or more nucleotides such that the discontinuous regions of the target RNA are not directly adjacent.

In some embodiments, the stem-loop sequence has a second portion that is reverse complementary to the first region or third region. In this embodiment, the second portion of the stem-loop hybridizes to the first or the third region and decreases the likelihood of non-specifically producing the output protein in the absence of the first target RNA. In some embodiments, the second portion of the stem-loop is contained in the loop sequence of the stem-loop sequence. When the stem-loop sequence has the first portion and the second portion, the length of the first portion combined with the length of the second portion amount to at most the full length or 100% of the stem-loop sequence. The second portion of the stem-loop sequence may be any portion of the stem-loop sequence. For instance, the second portion may be 1/10, 2/10, 3/10, 4/10, 5/10 or 1/2, 6/10, 7/10, 8/10, 9/10, 10/10, 1/4, or 3/4 of the stem-loop sequence or the loop sequence of the stem-loop sequence. The second portion may be 3 nucleotides, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 nucleotides, or more than 80 nucleotides. The second portion may be a range of nucleotides including, without limitation, 3-5 nucleotides, 5-10, 10-15, 15-20, 20-25, 25-30, 30-35, 35-40, 40-45, 45-50, 50-55, 55-60, 60-65, 65-70, 70-75, 75-80, 80-85, 10-20, 20-30, 30-40, 40-50, 50-60, 60-70, 70-80, etc.

When the sensor RNA contains a nucleotide sequence containing a stem-loop sequence containing an editable codon, the length of the stem-loop may have a specific length. For example, the stem-loop may be 4 nucleotides, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 nucleotides, or more than 90 nucleotides. The stem-loop sequence may be a range of nucleotides including, without limitation, 3-5 nucleotides, 5-10, 10-15, 15-20, 20-25, 25-30, 30-35, 35-40, 40-45, 45-50, 50-55, 55-60, 60-65, 65-70, 70-75, 75-80, 80-85, 10-20, 20-30, 30-40, 40-50, 50-60, 60-70, 70-80, etc.

Sensor RNAs containing a nucleotide sequence containing a stem-loop sequence containing an editable codon have certain advantages relative to sensor RNAs that do not contain such a stem-loop sequence, such as those disclosed in International Application PCT/US2022/033459. This is due to ADAR having separate domains for RNA editing (catalytic domain) and dsRNA binding. First, sensor RNAs containing a nucleotide sequence containing a stem-loop sequence containing an editable codon decouples the sequence that is being edited (e.g., a stop codon) from the sequence that recruits the ADAR protein (i.e., the dsRNA segment that is formed when the sensor nucleotide sequence hybridizes to the target RNA). Generally, if the editable codon in the sensor RNA is a UAG (stop codon) and there is only one mismatch in the stop codon relative to the target RNA then the target RNA should have a CCA sequence (or a sequence that hybridizes to a different stop codon having one mismatch with the stop codon). The presence of the CCA sequence (or an equivalent sequence for a different editable codon) potentially limits the number of possible target RNAs. Requiring a specific sequence (such as CCA or an equivalent sequence) to be present in the target RNA can be limiting because it restricts which subsequence a sensor could be created against; for example, a CCA or equivalent sequence may only present in highly structured parts of the target RNA, may only be present in the coding sequence, or may be present in protein-bound sections of a target RNA, all of which may contribute to lower availability for sensor-target hybridization, reducing efficiency. With a sensor containing a stem-loop, the range of suitable subsequences is greatly increased, so problematic target RNA subsequences can be avoided and efficient ones utilized instead. In some embodiments where gene fusions or splice variants are to be distinguished, flexibility in target RNA subsequence choice is needed, and is provided by the stem-loop design. Second, while ADAR editing is largely sequence-agnostic, there are some minor biases primarily driven by the catalytic domain which extend beyond the editable codon. Biases driven by the catalytic domain arc known in the art and have been described by, for example, Kuttan et al. (Proc Natl Acad Sci USA. 2012 Nov. 27;109 (48): E3295-304) which is specifically incorporated by reference herein. Editing sites in the sensor RNA may be dictated by the target RNA which precludes optimization of the editing site (i.e., the stop or non-stop codons of the present disclosure). By separating out the editing site from the sensor nucleotide sequence that hybridizes to the target RNA, to the editing site and the sensor nucleotide sequence can be optimized separately.

In some embodiments, the sensor RNA contains a non-start codon in place of a stop codon. In these embodiments, the sensor RNA contains the following: (i) a first nucleotide sequence containing a sensor nucleotide sequence that hybridizes to the target RNA, wherein the sensor nucleotide sequence contains a non-start codon (e.g. AUA) that contains one or more bases that are mismatched with the target RNA sequence, (ii) a second nucleotide sequence encoding a second cleavage domain, and (iii) a third nucleotide sequence encoding an output protein. In the presence of the target RNA, the sensor RNA hybridizes to the target RNA thereby forming a double stranded RNA molecule containing one or more base mismatches within the non-start codon or elsewhere. An ADAR protein then edits the adenosine base within the non-start codon (e.g. AUA to AUI) of the sensor RNA to an inosine base. This editing converts the non-start codon to a start codon which then allows the output protein to be produced from the sensor RNA within the biological sample.

In some embodiments, the sensor RNA has a start codon in place of a stop codon. In these embodiments, the sensor RNA has the following: (i) a first nucleotide sequence having a sensor nucleotide sequence that hybridizes to the target RNA, wherein the sensor nucleotide sequence has a start codon (e.g. AUG) that has one or more bases that are mismatched with the target RNA sequence and (ii) a second nucleotide sequence encoding a first cleavage domain, and (iii) a third nucleotide sequence encoding an output protein. In the presence of the target RNA, the sensor RNA hybridizes to the target RNA thereby forming a double-stranded RNA molecule having one or more base mismatches within the start codon or elsewhere. An ADAR protein then edits the adenosine base within the start codon (e.g., AUG to IUG) of the sensor RNA to an inosine base. This editing converts the start codon to a non-start codon which then inhibits the output protein from being produced from the sensor RNA within the cell.

In some cases, it is desired to reduce the immunogenicity or increase the half-life of the sensor RNA. Methods of reducing the immunogenicity of RNAs are known in the art such and have been described by, for example, Starostina et al. (Vaccines (Bascl). 2021 May 3;9 (5): 452) which is specifically incorporated by reference herein. In general, methods of reducing the immunogenicity or increasing the half-life of a sensor RNA involves the incorporation of modified ribonucleic acids into the sensor RNA. Modified ribonucleic acids that find use in the present disclosure includes, without limitation, pyridin-4-one ribonucleoside, 5-aza-uridine, 2-thio-5-aza-uridine, 2-thiouridine, 4-thio-pseudouridine, 2-thio-pseudouridine, 5-hy-droxyuridine, 3-methyluridine, 5-carboxymethyluridine, 1-carboxymethyl-pseudouridine, 5-propynyl-uridine, 1-propynyl-pseudouridine, 5-taurinomethyluridine, 1-taurinomethyl-pseudouridine, 5-taurinomethy 1-2-thiouridine, 1-tau-rinomethy 1-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thiodihydropseudouridine, 2-methoxyuridine, 2-methoxy-4-thiouridine, 4-methoxy-pseudouridine, 4-methoxy-2-thio-pseudouridine, 5-azacytidinc, pseudoisocytidinc, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2, 6-diaminopurine, 1-methyladenosine, N6-methyladenosine, N6-isopentenyladenosine, N6-(cis-hydroxyisopentenyadenosine, 2-methylthio-N-6-(cis-hydroxyisopentenyl) adenosine, N6-glycinylcarbamoyladenosine, N6-threonyl-carbamoyladenosine, 2-methylthio-N6-threonyl carbamoyladenosine, N6.N6-dimethyladenosine, 7-methyladenine, 2-methylthio-adenine, 2-methoxy-adenine, inosine, 1-me-thyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylgua-nosine, N2,N2-dimethylguanosine, 8-oxo-guanosine, 7-me-thyl-8-oxo-guanosine, 1-methyl-6-thio-guanosine, N2-methyl-6-thio-guano sine, N2,N2-dimethyl-6-thio-guanosine, methylcytosine, pseudouridine, methyladenosine, etc. In some embodiments, the pseudouridine is N1-methyl-pseudouridine. In some embodiments, the methyladenosine is a N6-methyladenosinc. In some embodiments, the methyladenosine is a N1-methyladenosine. In some embodiments, a portion of the nucleotides present in the sensor RNA are composed of modified ribonucleic acids. For instance, a portion of the uridines in the sensor RNA are replaced with pseudouridines. When the uridines of the sensor RNA are replaced with pseudouridines, a certain percentage of the uridines are replaced with pseudouridines. For instance, about 1-10%, about 10-20%, about 20-30%, about 30-40%, about 40-50%, about 50-60%, about 60-70%, about 70-80%, 80-90% or greater than 90% of the uridines are replaced with pseudouridines. In an embodiment, 75% or less of the uridines in the sensor RNAs are replaced with pseudouridines. In an embodiment, the sensor sequence does not have pseudouridines.

When a sensor RNA contains pseudouridines, the pseudouridine(s) may be in specific locations. In some embodiments, the pseudouridine(s) are not adjacent to adenosines that are the targets of ADAR editing. In some embodiments, the pseudouridine(s) are not contained in the sensor sequence that hybridizes with a target RNA. When a sensor RNA contains pseudouridines, the sensor may contain a particular stop codon. In some embodiments, the stop codon used is UGA. When the UGA stop codon is used, the adenosine in the UGA may be followed by a specific nucleotide. In some embodiments, the adenosine in the UGA is followed by guanosine such the nucleotide sequence is UGAG.

A marker protein of the present disclosure may be any marker protein that is useful for the detection of the presence of a sensor mRNA within a biological sample or a protein that may be used to normalized the signal of the output protein. For instance, the marker protein may be a fluorescent protein or a luminescent protein. Non-limiting examples of useful fluorescent proteins include but are not limited to GFP, EBFP, Azurite, Cerulean, mCFP, Turquoise, ECFP, mKeima-Red, TagCFP, AmCyan, mTFP, TurboGFP, TagGFP, EGFP, TagYFP, EYFP, Topaz, Venus, mCitrine, Turbo YFP, mOrange, TurboRFP, tdTomato, TagRFP, dsRed2, mRFP, mCherry, mPlum mRaspberry, mScarlet, etc. Examples of luminescent proteins, include without limitation, Cypridinia luciferase, Gaussia luciferase, Renilla luciferase, Phontinus luciferase, Luciola luciferase, Pyrophorus luciferase, Phrixothrix luciferase, a nanoluciferase, a nanoluciferase-PEST, etc.

In certain embodiments, the sensor RNA includes a nucleotide sequence that encodes a cleavage domain. Cleavage domains that find use in the present disclosure include without limitation, HIV-1 protease cleavage domain, TEV cleavage domain, preScission protease cleavage domain, HCV protease cleavage domain, RecA cleavage domain, self-cleaving domain, etc. When a self-cleaving domain is used then the self-cleaving domain may be a 2A self-cleaving domain. 2A self-cleaving domains that find use in the present disclosure include T2A, P2A, E2A, and F2A which are described in Szymczak-Workman et al. (Cold Spring Harb Protoc. 2012 Feb. 1;2012 (2): 199-204). In some embodiments, the sensor RNA includes a first and a second cleavage domain. When the sensor RNA includes a first and a second cleavage domain, the cleavage domains may be of the same type or they may be of a different type. For instance, the first cleavage domain may be a P2A self-cleaving domain and the second cleavage domain may also be a P2A self-cleaving domain or the first cleavage domain may be a P2A self-cleaving domain and the second cleavage domain may also be a T2A self-cleaving domain or any combination thereof.

Sensor RNAs that have sensor nucleotide sequences hybridizing to the 3′ or 5′ UTR have certain advantages relative to sensor RNAs that hybridize to coding sequences (CDS), such as those disclosed in International Application PCT/US2022/033459. First, ADAR editing is more efficient in the UTR when compared CDS because translating ribosomes may destabilize dsRNA. Second, RADAR is less likely to interfere with the production of the protein encoded by the target RNA because 1) dsRNA formation in the UTR rather than the CDS will not affect the translating ribosome, and 2) any bystander editing that occur in the UTR of the target RNA is less likely to cause detrimental outcomes because the coding sequence would not be edited.

The sensor nucleotide sequence of the present disclosure may be any length determined necessary for sufficient specificity to the target RNA. For instance, the sensor nucleotide sequence could be less than about 50 nucleotides, from about 50 to 60, about 60 to 70, about 70 to 80, about 80 to 90, about 90 to 100, about 100 to 110, about 110 to 120, about 120 to 130, about 130 to 140, about 140 to 150, about 150 to 160, about 160 to 170, about 170 to 180, about 180 to 190, about 190 to 200, about 200 to 210, about 210 to 220, about 220 to 230, about 230 to 240, about 240 to 250, about 250 to 260, about 260 to 270, about 270 to 280, about 280 to 290, about 290 to 300, about 300 to 310, about 310 to 320, about 320 to 330, about 330 to 340, about 340 to 350, about 350 to 360, about 360 to 370, about 370 to 380, about 380 to 390, about 390 to 400, about 400 to 410, about 410 to 420, about 420 to 430, about 430 to 440, about 440 to 450, about 450 to 460, about 460 to 470, about 470 to 480, about 480 to 490, about 490 to 500 or greater than 500 nucleotides in length.

When the sensor nucleotide sequence hybridizes to two non-contiguous regions within a target RNA, the distance between the two non-contiguous regions of the target may be any length. For instance, the distance between the two non-contiguous regions of the target may be less than about 50 nucleotides, from about 50 to 60, about 60 to 70, about 70 to 80, about 80 to 90, about 90 to 100, about 100 to 150, about 150 to 200, about 200 to 250, about 250 to 300, about 300 to 350, about 350 to 400, about 400 to 450, about 450 to 500 or greater than 500 nucleotides.

When the sensor nucleotide sequence hybridizes to two non-contiguous regions within the target RNA, the nucleotide sequence of the sensor nucleotide that hybridizes to the first region of the two non-contiguous regions with the target RNA may be any length. For instance, the nucleotide sequence of the sensor nucleotide that hybridizes to the first region of the two non-contiguous regions may be less than about 20 nucleotides, from about 20 to 30, about 30 to 40, about 40 to 50, about 50 to 60, about 60 to 70, about 70 to 80, about 80 to 90, about 90 to 100, about 100 to 110, about 110 to 120, about 120 to 130, about 130 to 140, about 140 to 150, about 150 to 160, about 160 to 170, about 170 to 180, about 180 to 190, about 190 to 200, about 200 to 210, about 210 to 220, about 220 to 230, about 230 to 240, about 240 to 250, about 250 to 260, about 260 to 270, about 270 to 280, about 280 to 290, about 290 to 300, about 300 to 310, about 310 to 320, about 320 to 330, about 330 to 340, about 340 to 350, about 350 to 360, about 360 to 370, about 370 to 380, about 380 to 390, about 390 to 400, about 400 to 410, about 410 to 420, about 420 to 430, about 430 to 440, about 440 to 450, about 450 to 460, about 460 to 470, about 470 to 480, about 480 to 490, about 490 to 500 or greater than 500 nucleotides in length.

When the sensor nucleotide sequence hybridizes to two non-contiguous regions with the target RNA, the nucleotide sequence of the sensor nucleotide that hybridizes to the second region of the two non-contiguous regions with the target RNA may be any length. For instance, the nucleotide sequence of the sensor nucleotide that hybridizes to the second region of the two non-contiguous regions may be less than about 20 nucleotides, from about 20 to 30, about 30 to 40, about 40 to 50, about 50 to 60, about 60 to 70, about 70 to 80, about 80 to 90, about 90 to 100, about 100 to 110, about 110 to 120, about 120 to 130, about 130 to 140, about 140 to 150, about 150 to 160, about 160 to 170, about 170 to 180, about 180 to 190, about 190 to 200, about 200 to 210, about 210 to 220, about 220 to 230, about 230 to 240, about 240 to 250, about 250 to 260, about 260 to 270, about 270 to 280, about 280 to 290, about 290 to 300, about 300 to 310, about 310 to 320, about 320 to 330, about 330 to 340, about 340 to 350, about 350 to 360, about 360 to 370, about 370 to 380, about 380 to 390, about 390 to 400, about 400 to 410, about 410 to 420, about 420 to 430, about 430 to 440, about 440 to 450, about 450 to 460, about 460 to 470, about 470 to 480, about 480 to 490, about 490 to 500 or greater than 500 nucleotides in length.

The sensor nucleotide sequence or the stem-loops of the present disclosure may include any stop or start codon including an adenosine residue. For example, the stop codon of the sensor nucleotide sequence may be UAG, UAA, or UGA. In general, the stop codons of the present disclosure are in-frame with the coding sequence of the output protein such that the output protein is produced when the stop codon is edited.

The output protein of the present disclosure may be any output protein desired. Examples of the output protein of the present disclosure include, without limitation, a fluorescent protein, a luminescent protein, a detectable protein, a transcription factor (e.g., a transcription factor that induces the transcription of a fluorescent protein), recombinase (e.g., a recombinase recombines DNA encoding DIO-fluorescent protein), etc. Non-limiting examples of useful fluorescent proteins include but are not limited to GFP, EBFP, Azurite, Cerulean, mCFP, Turquoise, ECFP, mKeima-Red, TagCFP, AmCyan, mTFP, TurboGFP, TagGFP, EGFP, TagYFP, EYFP, Topaz, Venus, mCitrine, Turbo YFP, mOrange, TurboRFP, tdTomato, TagRFP, dsRcd2, mRFP, mCherry, mPlum mRaspberry, mScarlet, etc. Examples of luminescent proteins, include without limitation, Cypridinia luciferase, Gaussia luciferase, Renilla luciferase, Phontinus luciferase, Luciola luciferase, Pyrophorus luciferase, Phrixothrix luciferase, a nanoluciferase, a nanoluciferase-PEST, etc.

The output protein may further include a tag to be used to detect the protein following its production. For instance, the tag may include, without limitation, a fluorescent protein, e.g., green fluorescent protein (GFP), YFP, RFP, CFP, mCherry, tdTomato, and the like; a histidine tag, e.g., a 6XHis tag; a hemagglutinin (HA) tag; a FLAG tag; a Myc tag; and the like.

Aspects of this disclosure include assaying for the presence of the output protein to assess the activity of the candidate agent to alter the target RNA. In some embodiments, the assaying for the output protein may contain using immunoblotting. In some embodiments, the assaying contains using microscopy. When the assaying contains microscopy the output protein may be conjugated to a fluorescent or luminescent protein or the output protein may be a fluorescent or luminescent protein. In some embodiments, the assaying for the presence of the output protein includes using flow cytometry. When the assaying includes flow cytometry, fluorescence-activating cell sorting may be used. In some embodiments, the assaying contains using a plate reader.

Assaying for the presence of the output protein is employed to determine if the candidate agent has activity to alter the target RNA. In some embodiments, the presence of the output protein is employed as indication that the candidate has activity in altering the target RNA in the cell. In some embodiments, the absence of the output protein is employed as an indication that the candidate has activity in altering the target RNA in the cell. In some embodiments, a change in the amount of the output protein is employed as an indication that the candidate has activity in altering the target RNA in the cell. In some embodiments, the change in the amount of the output protein is a decrease in the amount of the output protein. In some embodiments, the change in the amount of the output protein is an increase in the amount of the output protein.

In some embodiments, the method of screening a candidate agent for activity in altering a target RNA in a cell further contains contacting the cell with an ADAR protein or a coding sequence thereof. The ADAR protein may be any ADAR protein from any species. For instance, the ADAR protein may include without limitation, an ADAR (ADAR1), an ADAR p110, an ADAR p150, an ADAR2, an engineered ADAR protein such as a protein containing a deaminase domain of ADAR2 or a variant thereof and a MS2 RNA binding protein (MCP), an engineered ADAR protein that lacks a nuclear localization sequence, an engineered ADAR protein containing a nuclear export sequence, a modified ADAR comprising a nuclear localization signal, an engineered ADAR protein containing one or more dsRNA binding domains from one or more distinct ADAR proteins, an engineered ADAR protein containing a TAR RNA binding protein, an engineered ADAR protein containing a Lambda N peptide, a split engineered ADAR protein wherein the N and C terminus of the deaminase domain are produced separately and the two halves binding to one another in the presence of the target RNA, etc. Suitable engineered ADAR proteins have been described in Katrekar et al. (Nat Methods. 2019 March; 16 (3): 239-242.), Biswas et al. (iScience. 2020 Jul. 24;23 (7): 101318), Matthews et al. (Nat Struct Mol Biol. 2016 May;23 (5): 426-33), Cox et al. (Science. 2017 Nov. 24;358 (6366): 1019-1027) or Kuttan et al. (Proc Natl Acad Sci USA. 2012 Nov. 27;109 (48): E3295-304). Split engineered ADAR proteins are described in Katrekar et al. (Elife. 2022 Jan. 19;11: e75555). When the sensor RNA contains a start codon in place of a stop codon, a particular ADAR protein may be used. In some embodiments, the ADAR protein is ADAR2 when the sensor RNA contains a start codon in place of a stop codon.

In some embodiments, RNA editing proteins other than ADARs are used. For instance, proteins of the apolipoprotein B mRNA editing enzyme, catalytic polypeptide-like (APOBEC) family may be used. Examples of suitable APOBEC proteins include, without limitation, APOBEC1, APOBEC2, APOBEC3A, APOBEC3B, APOBEC3C, APOBEC3D, APOBEC3F, APOBEC3G, APOBEC3H, APOBEC4, etc.

Additional embodiments for using the sensor RNAs disclosed herein can be found in, for example, PCT application number US2023/063245 which is specifically incorporated by reference herein.

The present disclosure provides nucleic acids and recombinant vectors containing the sensor RNA. The recombinant vector includes, without limitation, a plasmid, a viral vector, a cosmid an artificial chromosome, etc. The cells of the present disclosure may be transfected prior to the contacting with the candidate agent. When transfection a cell is desired, vectors, such as plasmids viral vectors, cosmids, or artificial chromosomes, may be employed to engineer the cell to express the sensor RNA, as desired. Protocols of interest include those described in published PCT application WO1999/041258, the disclosure of which protocols are herein incorporated by reference.

Depending on the nature of the cell and/or expression construct, protocols of interest may include electroporation, particle gun technology, calcium phosphate precipitation, direct microinjection, viral infection, and the like. The choice of method is generally dependent on the type of cell being transformed and the circumstances under which the transformation is taking place (i.e., in vitro, ex vivo, or in vivo). A general discussion of these methods can be found in Ausubel, et al, Short Protocols in Molecular Biology, 3rd ed., Wiley & Sons, 1995. In some embodiments, lipofectamine and calcium-mediated gene transfer technologies are used. After the subject nucleic acids have been introduced into a cell, the cell may be incubated, normally at 37° C., sometimes under selection, for a period of about 1-24 hours in order to allow for the expression of the sensor RNA. In mammalian target cells, a number of viral-based expression systems may be utilized to express the sensor RNA(s). In cases where an adenovirus is used as an expression vector, the sensor RNA sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the chimeric protein in infected hosts. (e.g., see Logan & Shenk, Proc. Natl. Acad. Sci. USA 81:355-359 (1984)). The efficiency of expression may be enhanced by the inclusion of appropriate transcription enhancer elements, transcription terminators, etc. (see Bittner et al., Methods in Enzymol. 153:51-544 (1987)).

In some embodiments, the viral vector is a recombinant adeno-associated virus (AAV) vector. AAV vectors are DNA viruses of relatively small size that can integrate, in a stable and site specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

The application of AAV as a vector for gene therapy has been rapidly developed in recent years. Wild-type AAV can infect, with a comparatively high titer, dividing or non-dividing cells, or tissues of mammal, including human, and also can integrate into in human cells at specific site (on the long arm of chromosome 19) (Kotin ct al, Proc. Natl. Acad. Sci. U.S.A., 1990. 87:2211-2215; Samulski et al, EMBO J., 1991. 10:3941-3950 the disclosures of which are hereby incorporated by reference herein in their entireties). AAV vector without the rep and cap genes loses specificity of site-specific integration, but may still mediate long-term stable expression of exogenous genes. AAV vector exists in cells in two forms, wherein one is episomic outside of the chromosome; another is integrated into the chromosome, with the former as the major form. Moreover, AAV has not been found to be associated with any human disease, nor any change of biological characteristics arising from the integration has been observed. There are sixteen serotypes of AAV reported in literature, respectively named AAV1, AAV2, AAV3, AAV4, AAV5, AAV6, AAV7, AAV8, AAV9, AAV10, AAV11, AAV12, AAV13, AAV14, AAV15, and AAV16, wherein AAV5 is originally isolated from humans (Bantel-Schaal, and H. zur Hausen. Virology, 1984. 134:52-63), while AAV1-4 and AAV6 are all found in the study of adenovirus (Ursula Bantel-Schaal, Hajo Delius and Harald zur Hausen. J. Viral., 1999. 73:939-947).

AAV vectors may be prepared using any convenient methods. Adeno-associated viruses of any serotype are suitable (See, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. ME Bloom, RMLinden, C RParrish, Eds.) p 5-14, Rudder Arnold, London, U K (2006); and D E Bowles, J E Rabinowitz, R J Samulski “The Genus Dependovirus” (J R Kerr, SF Cotmore. ME Bloom, R M Linden, C R Parrish, Eds.) p 15-23, Rudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and W0/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCTIUS2005/027091, the disclosure of which is herein incorporated by reference in its entirety. The use of viral vectors derived from the AAVs for transferring genes in vitro and in vivo has been described (See e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some embodiments, the vector(s) for use in the methods of the invention are encapsidated into a virus particle (e.g. AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Accordingly, the invention includes a recombinant virus particle (recombinant because it contains a recombinant polynucleotide) comprising any of the vectors described herein. Methods of producing such particles are known in the art and are described in U.S. Pat. No. 6,596,535.

When the cell is transfected with a recombinant vector including the sensor RNA, the sensor RNA is operably linked to a promoter. Suitable promoters of the present disclosure include, without limitation, a SFFV promoter, a hEFla, a CMV promotor or a variant thereof, an inducible promoter, a CMV-tetO promoter, a tissue or cell specific promoter, etc.

### Kits

Also provided arc kits for practicing the methods described in the present disclosure. In general, subject kits may contain a cell containing a sensor RNA as described above. The cell may contain the target RNA. The cell may be in a homogenous collection of cells. The cell may be associated with a disease or condition. The cell may be in an organoid. The organoid may be an organoid associated with a disease or a condition. In some cases, the kit further contains an ADAR protein or a coding sequence thereof. When the kit contains a coding sequence of the ADAR it may be in a recombinant vector as described above. In some embodiments, the cell expresses the ADAR protein such as those described above. When the kit contains the ADAR protein or the coding sequence thereof, the ADAR protein may be any ADAR protein described above. In some embodiments, the kit contains a plasmid encoding the sensor RNA in the absence of the cell. In some embodiments, the kit contains the sensor RNA in the absence of the cell.

In some cases, the kit may further contain a positive and/or negative control. The positive control may be in the form of a candidate agent that alters the target RNA, a cell that constitutively expresses the output protein, or a combination thereof. The negative control may be in the form of a cell that does not contain the sensor RNA, a cell that does not contain the target RNA, a cell that does not contain an ADAR protein, a candidate agent that does not alter the target RNA, a sensor RNA that recognizes a RNA that is not the target RNA, or a combination thereof. When the absence of the output protein is employed as an indication the candidate has activity in altering the target RNA in the cell, the described positive controls may be negative controls and the described negative controls may be positive controls.

A subject kit can include any combination of components for performing the methods of the present disclosure. The components of a subject kit can be present as a mixture or can be separate entities. In some cases, the components are present as a liquid mixture. Components of a subject kit can be in the same or separate containers, in any combination.

The subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a remote site.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric. Standard abbreviations may be used, e.g., bp, base pair(s); kb, kilobasc(s); pl, picoliter(s); s or sec, second(s); min, minutc(s); h or hr, hour(s); aa, amino acid(s); nt, nucleotide(s); i.m., intramuscular (ly); i.p., intraperitoneal (ly); s.c., subcutaneous (ly); and the like.

### Example 1

### INTRODUCTION

The abundance of available single-cell transcriptomics data allows a cell's type and state to be inferred from its RNA signature. However, methods to detect and act upon RNA transcripts in living mammalian cells have historically been lacking. Such a tool could enable the identification and manipulation of specific cell types in living organisms as research tools, the selective ablation of pathogenic cells in human patients (e.g., cancer or autoimmune cells), and the cell type-specific delivery of in vivo gene therapies. Towards this goal, systems for RNA sensing have been described that leverage the RNA-editing abilities of adenosine deaminases acting on RNA (ADARs). These sensors take advantage of ADAR's ability to edit adenosines (A) to inosines (I), altering a stop codon upstream of a payload and allowing for the translation of a downstream payload conditioned upon the expression of a specific RNA transcript (referred to as “trigger”, “input”, or “target” hereafter).

Essential to these designs is a strong preference for specific sequence motifs in the trigger, analogous to CRISPR-Cas enzymes' requirements for a protospacer-adjacent motif (PAM) and here termed stop codon removal by ADAR motifs (“SCRAM”). When UAG is used as the stop codon, an appropriate SCRAM in the trigger is CCA. In the presence of a trigger, dsRNA forms around the stop codon, forming a C: A mismatch in the UAG: CCA pairing that increases editing efficiency. The dsRNA recruits ADAR, and the catalytic domain localizes to the site of the mismatch, editing the A in the UAG stop codon to I (read as guanine so that the codon UIG is read as UGG, encoding tryptophan instead of a stop) enabling translation of the payload.

Generally, a CCA subsequence within a transcript functions as the SCRAM, which base-pairs with UAG except for a C: A mismatch. Other subsequences may function as SCRAMs with reduced efficiency. Similarly to a PAM, the SCRAM requirement limits our ability to fully optimize sensor design (i.e., by prioritizing trigger regions with features such as low secondary structure or by empirically testing sensor candidates via tiling the trigger) and can prohibit the sensing of short transcripts or sub-sequences that lack this motif.

Here, SCRAM-less RNA sensors that are completely unconstrained by a particular sequence requirement are reported. Unlike previous-generation, “linear” RNA sensors that rely on ADAR editing dsRNA formed with the transcript of interest, this more modular system for RNA sensing using adenosine deaminases acting on RNA (“ModulADAR”) instead relies on ADAR editing a stem-loop sequence derived from natural ADAR substrates.

### Results

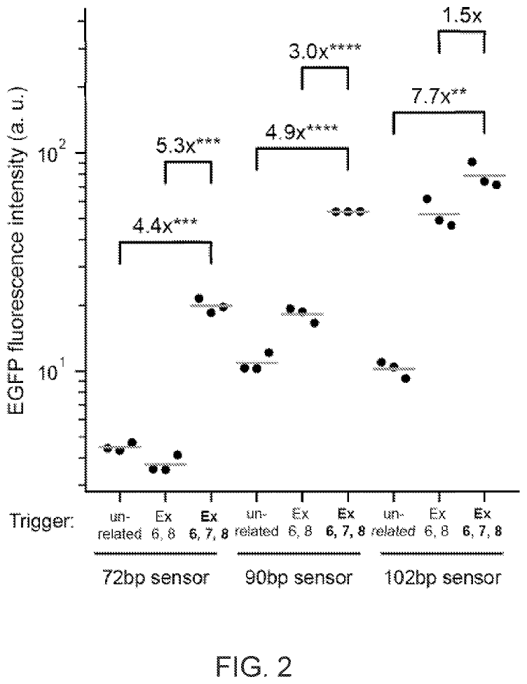

ModulADAR also has a unique advantage in sensing short transcripts or subsequences such as exons, enabling the discrimination of many splice isoforms previously inaccessible through “linear” RNA sensors. While 95% of genes undergo some form of alternative splicing, the short length of exons (on average <200 bp) makes it unlikely that a given exon will contain a SCRAM. One such exon is the exon 7 of SMN2 (survival of motor neuron 2). Alternative splicing of SMN2 to include exon 7 is of great clinical interest, as this isoform can rescue defects in SMN1 (survival of motor neuron 1) that cause spinal muscular atrophy (SMA). Sensors for exon 7 were designed, finding that a shorter sensor (72 bp) allowed for optimal discrimination between isoforms (FIG. 2). This is likely because longer sensors bind to longer regions in adjacent exons present in all splice isoforms and thus not contributing to discrimination.

TABLE 1
Sequences used in FIG. 2

| | | SEQ ID |
|---|---|---|
| Name | Sequence | NO: |
| Exon 6-8 | AUGGGAUCCAUAAUUCCCCCACCACCUCCCAUAUGUCCAGAUUCU- | 38 |
| trigger | CUUGAU- | |
| | GAUGCUGAUGCUUUGGGAAGUAUGUUAAUUUCAUGGUACAUGAGUG | |
| | GCUAUCAUACUGGCUAUUAUAUGGAAAUGCUGGCAUAGAGCAGCAC | |
| Exon 6-7-8 | AUGGGAUCCAUAAUUCCCCCACCACCUCCCAUAUGUCCAGAUUCU- | 39 |
| trigger | CUUGAU- | |
| | GAUGCUGAUGCUUUGGGAAGUAUGUUAAUUUCAUGGUACAUGAGUG | |
| | GCUAUCAUACUGGCUAUUAUAUGGGUUUUAGACAAAAUCAAAAA- | |
| | GAAGGAAGGUGCUCACAU- | |
| | UCCUUAAAUAUAAGGAGAAAUGCUGGCAUAGAGCAGCAC | |
| 72 bp sensor | AGCAUUUCUCCUUAUAUUGAAGGAAUGCGAGCAC- | 40 |
| | CUUGGCAUAUGCGACGGCCAUG- | |
| | GACUGGUUCAUAGCCGUCUGUUAUGUCCUUCUUUUUGAUUUUGUCU | |
| | AAAACCCAUAUAAUA | |
| 90 bp sensor | CUCUAUGCCAGCAUUUCUCCUUAUAUUGAAGGAAUGCGAGCAC- | 41 |
| | CUUGGCAUAUGCGAC- | |
| | GGCCAUGGACUGGUUCAUAGCCGUCUGUUAUGUCCUUCUUUUUGAU | |
| | UUUGUCUAAAACCCAUAUAAUAGCCAGUAUG | |
| 102 bp sensor | GUGCUGCUCUAUGCCAGCAUUUCUCCUUAUAUUGAAGGAAUGCGAG- | 42 |
| | CAC- | |
| | CUUGGCAUAUGCGACGGCCAUGGACUGGUUCAUAGCCGUCUGUUAU | |
| | GUCCUUCUUUUUGAUUUUGUCU- | |
| | AAAACCCAUAUAAUAGCCAGUAUGAUAGCC | |

### DISCUSSION

The ability of splice isoforms to both cause and ameliorate disease has motivated the development of splice-switching antisense-oligonucleotide (ASO) that work by selectively interfering with the splicing machinery. These include nusinersen (for the treatment of SMA) and several ASOs for the treatment of Duchenne muscular dystrophy (e.g., eteplisersen, golodirsen). With the development of more recent small molecules that can also selectively affect splicing (e.g., Risdiplam for the treatment of SMA), it has become apparent that more convenient and inexpensive small molecules can also selectively affect splicing. While risdiplam was discovered via the overexpression of an intron-exon cassette fused to a reporter gene (a strategy widely used for investigating alternative splicing), ModulADAR is better suited for screening both small molecules and ASOs as it allows for isoform sensing in the context of the native pre-mRNA. ModulADAR is better suited for developing patient-specific, “N of 1” therapies in patient-derived cells, as it does not require the targeted knock-in of an exon-intron reporter. This approach can be extended to the wide range of cancers and hereditary disorders caused by or potentially rescued by alternative splicing, including Dravet syndrome (caused by the inclusion of a “poison” exon in the sodium channel SCN1A) and Hutchinson-Gilford progeria syndrome (caused by defective splicing in the lamin gene LMNA).

### Example 2

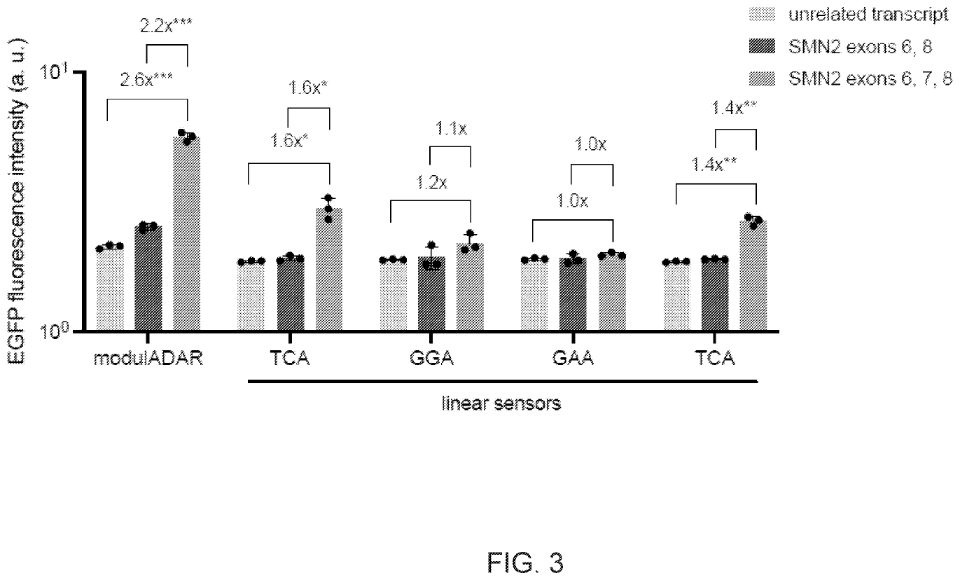

### Comparison Between ModulADAR and Linear Sensors Using Alternative Trinucleotides for Detecting SMN2 (FIG. 3)

WT Human Embryonic Kidney (HEK) 293 cells (ATCC, catalog no. CRL-1573) were cultured in a humidity-controlled incubator under standard culture conditions (37° C. with 5% CO2) in Dulbecco's Modified Eagle Medium, supplemented with 10% fetal bovine serum (Fisher Scientific, catalog no. FB12999102), 1 mM sodium pynvate (EMD Millipore, catalog no. TMS-005-C), 1×penicillin-streptomycin (Genesee, catalog no. 25-512), 2 mM 1-glutamine (Genesce, catalog no. 25-509) and 1× MEM non-essential amino acids (Genesee, catalog no. 25-536). HEK 293 cells were cultured in 24-well tissue culture-treated plates under standard culture conditions. When cells were 70-90% confluent, the cells were transiently transfected with plasmid constructs using the jetOPTIMUS DNA transfection Reagent (Polyplus catalog #117-15), as per manufacturer's instructions using 0.375 μL of reagent per 50 μL of jetOPTIMUS buffer for 500 ng total DNA transfections in the 24-well format. Cells were harvested approximately 48 hours after transfection by trypsinization and resuspended in flow buffer (HBSS+2.5 mg/mL bovine serum albumin). Post 40 μm straining, cells were analyzed by flow cytometry (Biorad ZES Cell Analyzer), and data was processed with the cytoflow Python package. For experiments comparing two groups, a Bonferroni-corrected two-tailed Student t-test was used to assess significance.

Cells were transfected with 200 ng plasmid DNA encoding the sensor sequence. 30 ng plasmid DNA encoding ADARp150, and 270 ng plasmid DNA encoding either an unrelated transcript; SMN2 exon 6, a portion of exon 8, and out-of-frame nanoluciferase; or human SMN2 exon 6, exon 7, a portion of exon 8, and in-frame nanoluciferase,

TABLE 1
Sensor nucleotides and target RNAs used in Example 2

| | plasmid | | | SEQ ID |
|---|---|---|---|---|
| | designation | plasmid name | sensor or trigger sequence | NO: |
| SMN2 exons | NSK162 | CMV-TO-SMN2 | ATGGGATCCATAATTCCCCCACCAC- | 13 |
| 6, 8 | | minigene Nluc- | CTCCCATATGTCCAGATTCTCTT- | |
| | | inactive | GATGATGCTGATGCTTTGGGAAGTATGTT | |
| | | (exon 6, 8) | AATTTCATGGTACATGAGTGGCTATCAT- | |
| | | | ACTGGCTATTATATGGAAATGCTGG- | |
| | | | CATAGAG- | |
| | | | CAGCACGTCTTCACACTCGAAGATTTCGT | |
| | | | TGGGGACTGGCGACAGACAGCCGGC- | |
| | | | TACAACCTGGACCAAGTCCTT- | |
| | | | GAACAGGGAGGTGTGTCCAGTTTGTTTC | |
| | | | AGAATCTCGGGGTGTCCG- | |
| | | | TAACTCCGATCCAAAGGATTGTCCTGAG | |
| | | | CGGTGAAAATGGGCTGAA- | |
| | | | GATCGACATCCATGTCATCATCCCG- | |
| | | | TATGAAGGTCTGAGCGGCGACCAAATGG | |
| | | | GCCAGATCGAAAAAATTTTTAAGGTGGT | |
| | | | GTACCCTGTGGATGATCATCAC- | |
| | | | TTTAAGGTGATCCTGCACTATGGCACAC- | |
| | | | TGGTAATCGAC- | |
| | | | GGGGTTACGCCGAACATGATCGACTATT | |
| | | | TCGGACGGCCG- | |
| | | | TATGAAGGCATCGCCGTGTTCGACGG- | |
| | | | CAAAAAGATCACTG- | |
| | | | TAACAGGGACCCTGTGGAACGGCAACAA | |
| | | | AATTATCGAC- | |
| | | | GAGCGCCTGATCAACCCCGAC- | |
| | | | GGCTCCCTGCTGTTCCGAGTAAC- | |
| | | | CATCAAC- | |
| | | | GGAGTGACCGGCTGGCGGCTGTGCGAAC | |
| | | | GCATTCTGGCGTAG | |
| SMN2 exons | NSK163 | CMV-TO-SMN2 | ATGGGATCCATAATTCCCCCACCAC- | 14 |
| 6, 7, 8 | | minigene Nluc- | CTCCCATATGTCCAGATTCTCTT- | |
| | | active | GATGATGCTGATGCTTTGGGAAGTATGTT | |
| | | (exon 6, 7, 8) | AATTTCATGGTACATGAGTGGCTATCAT- | |
| | | | ACTGGCTATTATATGGGTTTTAGA- | |
| | | | CAAAATCAAAAAGAAGGAAGGTGCTCAC | |
| | | | ATTCCTTAAATATAAGGAGAAATGCTGG- | |
| | | | CATAGAGCAGCACGTCTTCACACTCGAA- | |
| | | | GAT- | |
| | | | TTCGTTGGGGACTGGCGACAGACAGCCG | |
| | | | GCTACAACCTGGACCAAGTCCTT- | |
| | | | GAACAGGGAGGTGTGTCCAGTTT- | |
| | | | GTTTCAGAATCTCGGGGTGTCCGTAACTC | |
| | | | CGATCCAAAGGATTGTCCTGAGCGGTGA | |
| | | | AAATGGGCTGAA- | |
| | | | GATCGACATCCATGTCATCATCCCG- | |
| | | | TATGAAGGTCTGAGCGGCGAC- | |
| | | | CAAATGGGCCAGATCGAAAAAATTTTTA | |
| | | | AGGTGGTGTACCCTGTG- | |
| | | | GATGATCATCACTTTAAGGTGATCCTG- | |
| | | | CACTATGGCACACTGG- | |
| | | | TAATCGACGGGGTTACGCCGAACATGAT | |
| | | | CGACTATTTCGGACGGCCG- | |
| | | | TATGAAGGCATCGCCGTGTTCGACGG- | |
| | | | CAAAAAGATCACTG- | |
| | | | TAACAGGGACCCTGTGGAACGGCAACAA | |
| | | | AATTATCGAC- | |
| | | | GAGCGCCTGATCAACCCCGAC- | |
| | | | GGCTCCCTGCTGTTCCGAGTAAC- | |
| | | | CATCAAC- | |
| | | | GGAGTGACCGGCTGGCGGCTGTGCGAAC | |
| | | | GCATTCTGGCGTAG | |
| modulADAR | NSK173 | SFFV-mCherry- | AGCATTTCTCCTTA- | 15 |
| | | SGc(Smn2ex7.os13- | TATTGAAGGAATGCGAGCACCTTGGCAT- | |
| | | 72 bp)cSG-EGFP- | ATGCGAC- | |
| | | (3xMS2)(FLP-IN) | GGCCATGGACTGGTTCATAGCCGTCTGTT | |
| | | | ATGTCCTTCTTTTTGATTTT- | |
| | | | GTCTAAAACCCATATAATA | |
| TCA linear | NSK204 | SFFV-mCherry- | CTTATATTTAAGGAATGTGAGCAC- | 16 |
| sensor | | SGc(SNM2.scram1) | CTTCCTTCTTTTTAGTTTT- | |
| | | cSG-EGFP- | GTCTAAAACCCATATCATGGCCAGTATG | |
| | | (3xMS2)(FLP-IN) | G | |
| GGA linear | NSK205 | SFFV-mCherry- | AGCATTTCTCCTTATATTTCAG- | 17 |
| sensor | | SGc(SNM2.scram2) | GAATGTGGGCACCTTAGTTCTTTTTGAT- | |
| | | cSG-EGFP- | TTTGTCTAAAACCCATATAATA | |
| | | (3xMS2)(FLP-IN) | | |
| GAA linear | NSK206 | SFFV-mCherry- | CAGCATTTCTCCTTA- | 18 |
| sensor | | SGc(SNM2.scram3) | TATTTAAGGAATGTGAGCAC- | |
| | | CSG-EGFP- | CTAGCTTCTTTTTGATTTT- | |
| | | (3xMS2)(FLP-IN) | GTCTCAAACCCATATAAT | |
| TCA linear | NSK206 | SFFV-mCherry- | TATGCCAGCATTTCTCCTTATATTTCAG- | 19 |
| sensor | | SGc(SNM2.scram4) | GAATGTAGGCACCTTCCTTCTTTTTGAT- | |
| | | CSG-EGFP- | TTTGTCTAAAACCCAT | |
| | | (3xMS2)(FLP-IN) | | |

### Example 3

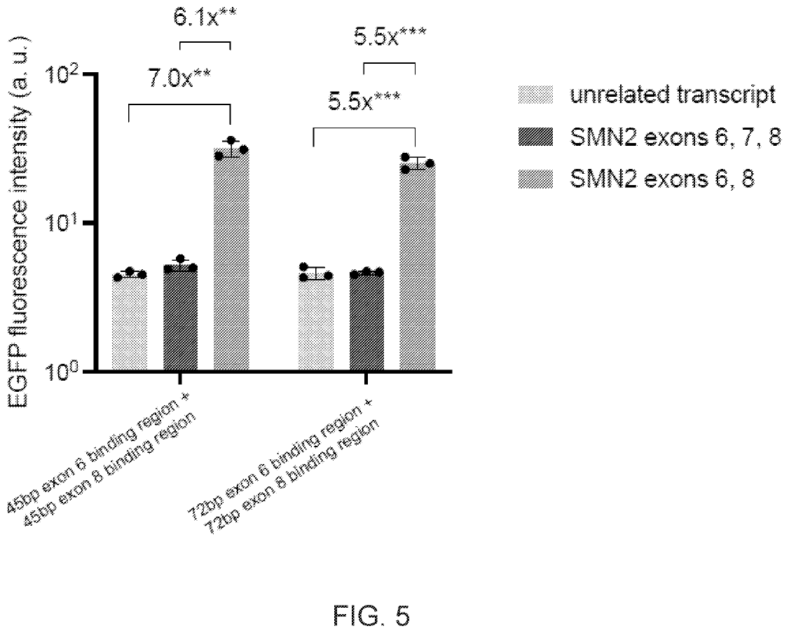

### Comparison of Different Lengths of Exon 6 and Exon 8 Binding Regions for SMN2 (FIG. 5)

Cells were transfected with 200 ng plasmid DNA encoding the sensor sequence, 30 ng plasmid DNA encoding ADARp150, and 270 ng plasmid DNA encoding either an unrelated transcript; SMN2 exon 6, a portion of exon 8, and out-of-frame nanoluciferase; or human SMN2 exon 6, exon 7, a portion of exon 8, and in-frame nanoluciferase.

TABLE 2
Sensor nucleotides and target RNAs used in Example 3

| | plasmid | | | SEQ ID |
|---|---|---|---|---|
| | designation | plasmid name | sensor sequence | NO: |
| SMN2 | NSK162 | CMV-TO-SMN2 | ATGGGATCCA- | 13 |
| exons 6, 8 | | minigene Nluc- | TAATTCCCCCACCAC- | |
| | | inactive (exon 6, 8) | CTCCCATATGTCCAGAT- | |
| | | | TCTCTT- | |
| | | | GATGATGCTGATGCTTTGGGAA | |
| | | | GTATGTTAATTTCATGG- | |
| | | | TACATGAGTGGCTATCAT- | |
| | | | ACTGGCTATTA- | |
| | | | TATGGAAATGCTGG- | |
| | | | CATAGAGCAGCACGTCTTCACA | |
| | | | CTCGAAGATTTCGTTGGG- | |
| | | | GACTGGCGACAGACAGCCGGC- | |
| | | | TACAACCTGGACCAAGTCCTT- | |
| | | | GAACAGGGAGGTGTGTCCAGT | |
| | | | TTGTTTCAGAATCTCGGGGTGT | |
| | | | CCG- | |
| | | | TAACTCCGATCCAAAGGATTGT | |
| | | | CCTGAGCGGTGAAAATGGGCT | |
| | | | GAA- | |
| | | | GATCGACATCCATGTCATCATC | |
| | | | CCG- | |
| | | | TATGAAGGTCTGAGCGGCGAC- | |
| | | | CAAATGGGCCAGATCGAAAAA | |
| | | | ATTTTTAAGGTGGTG- | |
| | | | TACCCTGTGGATGATCATCAC- | |
| | | | TTTAAGGTGATCCTGCAC- | |
| | | | TATGG- | |
| | | | CACACTGGTAATCGACGGGGTT | |
| | | | ACGCCGAACATGATCGAC- | |
| | | | TATTTCGGACGGCCG- | |
| | | | TATGAAGGCATCGCCGTGTTCG | |
| | | | ACGGCAAAAAGATCACTG- | |
| | | | TAACAGGGACCCTGTGGAAC- | |
| | | | GGCAACAAAATTATCGAC- | |
| | | | GAGCGCCTGATCAACCCCGAC | |
| | | | GGCTCCCTGCTGTTCCGAG- | |
| | | | TAACCATCAACGGAGTGAC- | |
| | | | CGGCTGGCGGCTGTGCGAAC- | |
| | | | GCATTCTGGCGTAG | |
| SMN2 | NSK163 | CMV-TO-SMN2 | ATGGGATCCA- | 14 |
| exons 6, | | minigene Nluc-active | TAATTCCCCCACCAC- | |
| 7, 8 | | (exon 6, 7, 8) | CTCCCATATGTCCAGAT- | |
| | | | TCTCTT- | |
| | | | GATGATGCTGATGCTTTGGGAA | |
| | | | GTATGTTAATTTCATGG- | |
| | | | TACATGAGTGGCTATCAT- | |
| | | | ACTGGCTATTA- | |
| | | | TATGGGTTTTAGA- | |
| | | | CAAAATCAAAAAGAAGGAAGG | |
| | | | TGCTCACATTCCTTAAA- | |
| | | | TATAAGGAGAAATGCTGG- | |
| | | | CATAGAGCAGCAC- | |
| | | | GTCTTCACAC- | |
| | | | TCGAAGATTTCGTTGGGGACTG | |
| | | | GCGACAGACAGCCGGC- | |
| | | | TACAACCTGGACCAAGTCCTT- | |
| | | | GAACAGGGAGGTGTGTCCAG- | |
| | | | TTT- | |
| | | | GTTTCAGAATCTCGGGGTGTCC | |
| | | | GTAACTCCGATCCAAAGGATTG | |
| | | | TCCTGAGCGGTGAAAATGGGCT | |
| | | | GAA- | |
| | | | GATCGACATCCATGTCATCATC | |
| | | | CCG- | |
| | | | TATGAAGGTCTGAGCGGCGAC- | |
| | | | CAAATGGGCCAGATCGAAAAA | |
| | | | ATTTTTAAGGTGGTG- | |
| | | | TACCCTGTGGATGATCATCAC- | |
| | | | TTTAAGGTGATCCTGCAC- | |
| | | | TATGG- | |
| | | | CACACTGGTAATCGACGGGGTT | |
| | | | ACGCCGAACATGATCGAC- | |
| | | | TATTTCGGACGGCCG- | |
| | | | TATGAAGGCATCGCCGTGTTCG | |
| | | | ACGGCAAAAAGATCACTG- | |
| | | | TAACAGGGACCCTGTGGAAC- | |
| | | | GGCAACAAAATTATCGAC- | |
| | | | GAGCGCCTGATCAACCCCGAC | |
| | | | GGCTCCCTGCTGTTCCGAG- | |
| | | | TAACCATCAACGGAGTGAC- | |
| | | | CGGCTGGCGGCTGTGCGAAC- | |
| | | | GCATTCTGGCGTAG | |
| 45 bp + | NSK241 | SFFV-mCherry- | CGAAATCTTCGAGTGTGGA- | 20 |
| 45 bp | | SGc(os13.NOT- | GACGTGCTGCTCTATGCCAG- | |
| | | SMN2.45, 55, 45)cSG- | CATTTCTGGCATATGCGAC- | |
| | | EGFP-(3xMS2)(FLP-IN) | GGCCATGGATCCTTATATTTAA | |
| | | | GGAATGTGAGCAC- | |
| | | | CTTCCTTCTTTTTGGTTTT- | |
| | | | GTCTAAAAC- | |
| | | | CTTCATAGCCGTCTGTTATGCA | |
| | | | TATAATAGCCAGTATGA- | |
| | | | TAGCCACTCATGTAC- | |
| | | | CATGGAATTCAA | |
| 36 bp + | NSK242 | SFFV-mCherry- | CGAGTGTGGAGAC- | 21 |
| 36 bp | | SGc(os13.NOT- | GTGCTGCTCTATGCCAG- | |
| | | SMN2.36, 55, 36)cSG- | CATTTCTGGCATATGCGAC- | |
| | | EGFP-(3xMS2)(FLP-IN) | GGCCATGGATCCTTATATTTAA | |
| | | | GGAATGTGAGCAC- | |
| | | | CTTCCTTCTTTTTGGTTTT- | |
| | | | GTCTAAAAC- | |
| | | | CTTCATAGCCGTCTGTTATGCA | |
| | | | TATAATAGCCAGTATGA- | |
| | | | TAGCCACTCATGTACCA | |

### Example 4

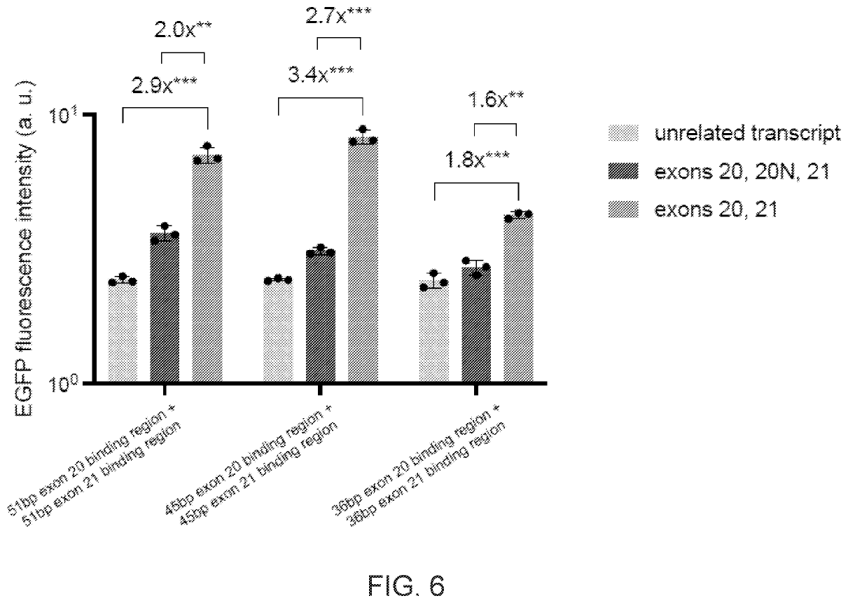

### Comparison of Different Lengths of Exon 20 and Exon 21 Binding Regions for SNC1a (FIG. 6)

Cells were transfected with 200 ng plasmid DNA encoding the sensor sequence; 30 ng plasmid DNA encoding ADARp150; and 270 ng plasmid DNA encoding either an unrelated transcript, SONIA exons 20, 20N, 21, or SCN1A exons 20, 21.

TABLE 3
Sensor nucleotides and target RNAs used in Example 4

| | plasmid | | | SEQ ID |
|---|---|---|---|---|
| | designation | plasmid name | sensor or trigger sequence | NO: |
| exons 20, | NSK250 | CMV-TO- | ATGGTTTCATTGGTCAGTTTAACAG- | 22 |
| 20N, 21 | | SCN1A(exons | CAAATGCCTTGGGTTACTCAGAACTT- | |
| | | 20, 20N, 21) | GGAGCCATCAAATCTCTCAGGACACTAAGA | |
| | | | GCTCTGAGACCTCTAA- | |
| | | | GAGCCTTATCTCGATTT- | |
| | | | GAAGGGATGAGGGATAATCTT- | |
| | | | GCTCCAACTTGGATGGGGTGGAGCGCTGGT | |
| | | | TCCTCCCCTGAGCCCTTTATTATGGGTGGTT- | |
| | | | GTGAATGCCCTTTTAGGAG- | |
| | | | CAATTCCATCCATCATGAATGTGCTTCTGGT | |
| | | | TTGTCTTATATTCTGGCTAATTTTCAG- | |
| | | | CATCATGGGCGTAAATTTGTTTGCTGG- | |
| | | | CAAATTC- | |
| | | | TACCACTGTATTAACACCACAACTGGTGAC | |
| | | | AGGTTTGACATCGAAGACGTGAATAATCAT- | |
| | | | ACTGATTGCCTAAAACTAATAGAAA- | |
| | | | GAAATGA- | |
| | | | GACTGCTCGATGGAAAAATGTGAAAGTAAA | |
| | | | CTTTGATAATGTAGGATTTGGG- | |
| | | | TATCTCTCTTTGCTTCAAGTTTGTGA | |
| exons 20, | NSK249 | CMV-TO- | ATGGTTTCATTGGTCAGTTTAACAG- | 23 |
| 21 | | SCN1A (exons | CAAATGCCTTGGGTTACTCAGAACTT- | |
| | | 20, 21) | GGAGCCATCAAATCTCTCAGGACACTAAGA | |
| | | | GCTCTGAGACCTCTAA- | |
| | | | GAGCCTTATCTCGATTT- | |
| | | | GAAGGGATGAGGGTGGTT- | |
| | | | GTGAATGCCCTTTTAGGAGCAATTCCATCCA | |
| | | | TCATGAATGTGCTTCTGGTTTGTCTTA- | |
| | | | TATTCTGGCTAATTTTCAGCATCATGGGCG- | |
| | | | TAAATTTGTTTGCTGGCAAATTCTACCACTG | |
| | | | TATTAACACCACAACTGGTGACAGGTTT- | |
| | | | GACATCGAAGACGTGAATAATCAT- | |
| | | | ACTGATTGCCTAAAACTAATAGAAAGAAAT | |
| | | | GA- | |
| | | | GACTGCTCGATGGAAAAATGTGAAAGTAAA | |
| | | | CTTTGATAATGTAGGATTTGGG- | |
| | | | TATCTCTCTTTGCTTCAAGTTTGA | |
| 51 bp + | NSK251 | SFFV-mCherry- | AAGCACATTCATGATGGATGGAATT- | 24 |
| 51 bp | | SGc(os13.NOT- | GCTCCTCAAAGGGCATTCACAACCACTGG- | |
| | | SCN1A_20N.51, | CAT- | |
| | | 64, 51)cSG- | ATGCGACGGCCATGGACCATAATAAAGGGC | |
| | | EGFP-(3xMS2) | TCAGGGGAGGAAC- | |
| | | (FLP-IN) | CAGCGCTCCACCCCATCCAAGTTGGAGCAA- | |
| | | | GAT- | |
| | | | TATCTTCATAGCCGTCTGTTATGCCTCATCC | |
| | | | CTTCAAATCGAGATCAGGCTCTT- | |
| | | | GGAGGTCTCAGAGCTCTTGG | |
| 45 bp + | NSK252 | SFFV-mCherry- | ATTCATGATGGATGGAATT- | 25 |
| 45 bp | | SGc(os13.NOT- | GCTCCTCAAAGGGCATTCACAACCACTGG- | |
| | | SCN1A_20N.45, | CATATGCGAC- | |
| | | 64, 45)cSG- | GGCCATGGACCATAATAAAGGGCTCAGGGG | |
| | | EGFP-(3xMS2) | AGGAACCAGCGCTCCACCCCATCCAAGTT- | |
| | | (FLP-IN) | GGAGCAAGAT- | |
| | | | TATCTTCATAGCCGTCTGTTATGCCTCATCC | |
| | | | CTTCAAATCGAGATCAGGCTCTT- | |
| | | | GGAGGTCTCAGAGC | |
| 36 bp + | NSK253 | SFFV-mCherry- | GGATGGAATTGCTCCTCAAAGGG- | 26 |
| 36 bp | | SGc(os13.NOT- | CATTCACAACCACTGGCATATGCGAC- | |
| | | SCN1A_20N.36, | GGCCATGGACCA- | |
| | | 64, 36)cSG- | TAATAAAGGGCTCAGGGGAGGAACCAGCG | |
| | | EGFP-(3xMS2) | CTCCACCCCATCCAAGTTGGAGCAAGAT- | |
| | | (FLP-IN) | TATCTTCATAGCCGTCTGTTATGCCTCATCC | |
| | | | CTTCAAATCGAGATCAGGCTCTTGGAGG | |

### Example 5

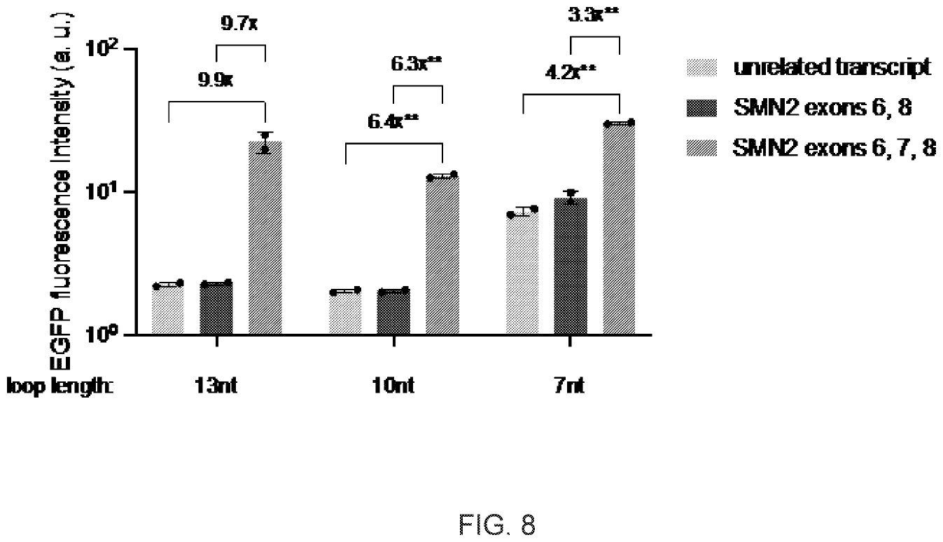

### Comparison of Different Lengths of Stem-Loops in Low Background Sensor RNAs for SMN2 (FIG. 8)

Cells were transfected with 200 ng plasmid DNA encoding the sensor sequence, 30 ng plasmid DNA encoding ADARp150, and 270 ng plasmid DNA encoding either an unrelated transcript; SMN2 exon 6, a portion of exon 8, and out-of-frame nanoluciferase; or human SMN2 exon 6, exon 7, a portion of exon 8, and in-frame nanoluciferase.

TABLE 4
Sensor nucleotides and target RNAs used in Example 5

| | plasmid | | | SEQ ID |
|---|---|---|---|---|
| | designation | plasmid name | sensor sequence | NO: |
| SMN2 | NSK162 | CMV-TO-SMN2 | ATGGGATCCATAATTCCCCCACCAC- | 13 |
| 6, 8 | | minigene Nluc- | CTCCCATATGTCCAGATTCTCTT- | |
| | | inactive | GATGATGCTGATGCTTTGGGAAGTATGTTA | |
| | | (exon 6, 8) | ATTTCATGGTACATGAGTGGCTATCAT- | |
| | | | ACTGGCTATTATATGGAAATGCTGGCATA- | |
| | | | GAGCAG- | |
| | | | CACGTCTTCACACTCGAAGATTTCGTTGGG | |
| | | | GACTGGCGACAGACAGCCGGCTACAAC- | |
| | | | CTGGACCAAGTCCTT- | |
| | | | GAACAGGGAGGTGTGTCCAG- | |
| | | | TTTGTTTCAGAATCTCGGGGTGTCCGTAAC | |
| | | | TCCGATCCAAAGGATTGTCCTGAGCGGTGA | |
| | | | AAATGGGCTGAA- | |
| | | | GATCGACATCCATGTCATCATCCCG- | |
| | | | TATGAAGGTCTGAGCGGCGAC- | |
| | | | CAAATGGGCCAGATCGAAAAAATTTTTAA | |
| | | | GGTGGTGTACCCTGTGGATGATCATCAC- | |
| | | | TTTAAGGTGATCCTGCACTATGGCACAC- | |
| | | | TGG- | |
| | | | TAATCGACGGGGTTACGCCGAACATGATC | |
| | | | GACTATTTCGGACGGCCG- | |
| | | | TATGAAGGCATCGCCGTGTTCGACGG- | |
| | | | CAAAAAGATCACTG- | |
| | | | TAACAGGGACCCTGTGGAACGGCAACAAA | |
| | | | ATTATCGAC- | |
| | | | GAGCGCCTGATCAACCCCGAC- | |
| | | | GGCTCCCTGCTGTTCCGAGTAAC- | |
| | | | CATCAAC- | |
| | | | GGAGTGACCGGCTGGCGGCTGTGCGAACG | |
| | | | CATTCTGGCGTAG | |
| SMN2 | NSK163 | CMV-TO-SMN2 | ATGGGATCCATAATTCCCCCACCAC- | 14 |
| 6, 7, 8 | | minigene Nluc- | CTCCCATATGTCCAGATTCTCTT- | |
| | | active | GATGATGCTGATGCTTTGGGAAGTATGTTA | |
| | | (exon 6, 7, 8) | ATTTCATGGTACATGAGTGGCTATCAT- | |
| | | | ACTGGCTATTATATGGGTTTTAGA- | |
| | | | CAAAATCAAAAAGAAGGAAGGTGCTCACA | |
| | | | TTCCTTAAATATAAGGAGAAATGCTGG- | |
| | | | CATAGAGCAGCACGTCTTCACACTCGAA- | |
| | | | GAT- | |
| | | | TTCGTTGGGGACTGGCGACAGACAGCCGG | |
| | | | CTACAACCTGGACCAAGTCCTT- | |
| | | | GAACAGGGAGGTGTGTCCAGTTT- | |
| | | | GTTTCAGAATCTCGGGGTGTCCGTAACTCC | |
| | | | GATCCAAAGGATTGTCCTGAGCGGTGAAA | |
| | | | ATGGGCTGAA- | |
| | | | GATCGACATCCATGTCATCATCCCG- | |
| | | | TATGAAGGTCTGAGCGGCGAC- | |
| | | | CAAATGGGCCAGATCGAAAAAATTTTTAA | |
| | | | GGTGGTGTACCCTGTGGATGATCATCAC- | |
| | | | TTTAAGGTGATCCTGCACTATGGCACAC- | |
| | | | TGG- | |
| | | | TAATCGACGGGGTTACGCCGAACATGATC | |
| | | | GACTATTTCGGACGGCCG- | |
| | | | TATGAAGGCATCGCCGTGTTCGACGG- | |
| | | | CAAAAAGATCACTG- | |
| | | | TAACAGGGACCCTGTGGAACGGCAACAAA | |
| | | | ATTATCGAC- | |
| | | | GAGCGCCTGATCAACCCCGAC- | |
| | | | GGCTCCCTGCTGTTCCGAGTAAC- | |
| | | | CATCAAC- | |
| | | | GGAGTGACCGGCTGGCGGCTGTGCGAACG | |
| | | | CATTCTGGCGTAG | |
| 13 nt | NSK467 | SFFV-mCherry- | AGCATTTCTCCTTA- | 27 |
| | | SGc(Smn2ex7.os | TATTGAAGGAATGCGAGCACCTTGGCAT- | |
| | | 13- | ATGCGAC- | |
| | | 72 bp.lowBG.13 bp | GGCCATGGAAGGTGCTCGCATTTTCATAGC | |
| | | loop)cSG- | CGTCTGTTATGTCCTTCTTTTTGATTTT- | |
| | | EGFP-(3xMS2) | GTCTAAAACCCATATAATA | |
| | | (FLP-IN) | | |
| 10 nt | NSK468 | SFFV-mCherry- | AGCATTTCTCCTTA- | 28 |
| | | SGc(Smn2ex7.os | TATTGAAGGAATGCGAGCACCTTGGCAT- | |
| | | 13- | ATGCGAC- | |
| | | 72 bp.lowBG.10 bp | GGCCATGGAAGGTGCTCGCTTCATAGCCGT | |
| | | loop)cSG- | CTGTTATGTCCTTCTTTTTGATTTT- | |
| | | EGFP-(3xMS2) | GTCTAAAACCCATATAATA | |
| | | (FLP-IN) | | |
| 7 nt | NSK469 | SFFV-mCherry- | AGCATTTCTCCTTA- | 29 |
| | | SGc(Smn2ex7.os | TATTGAAGGAATGCGAGCACCTTGGCAT- | |
| | | 13- | ATGCGAC- | |
| | | 72 bp.lowBG.7 bp | GGCCATGGAAGGTGCTTTCATAGCCGTCTG | |
| | | loop)cSG-EGFP- | TTATGTCCTTCTTTTTGATTTT- | |
| | | (3xMS2)(FLP-IN) | GTCTAAAACCCATATAATA | |

### Example 6

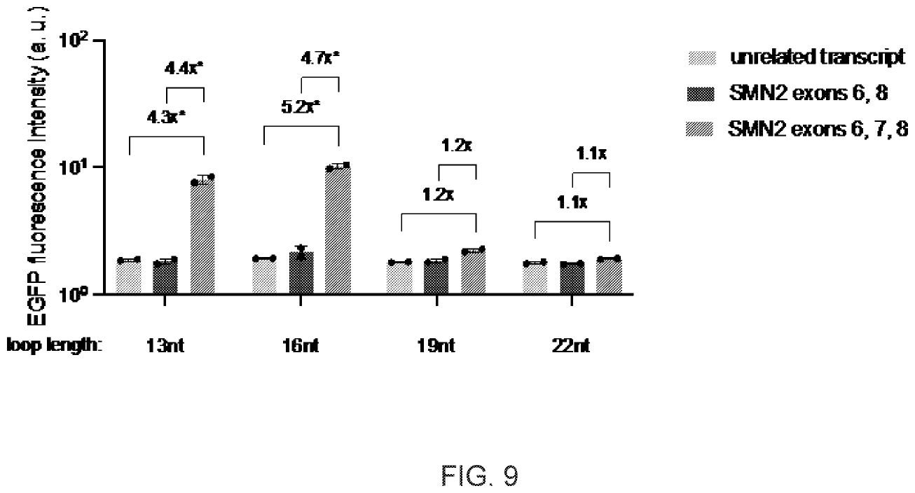

### Comparison of Different Lengths of Stem-Loops in Low Background Sensor RNAs for SMN2 (FIG. 9)

Cells were transfected with 200 ng plasmid DNA encoding the sensor sequence, 30 ng plasmid DNA encoding ADARp150, and 270 ng plasmid DNA encoding either an unrelated transcript; SMN2 exon 6, a portion of exon 8, and out-of-frame nanoluciferase; or human SMN2 exon 6, exon 7, a portion of exon 8, and in-frame nanoluciferase.

TABLE 5
Sensor nucleotides and target RNAs used in Example 6

| | plasmid | | | SEQ ID |
|---|---|---|---|---|
| | designation | plasmid name | sensor sequence | NO: |
| SMN2 | NSK162 | CMV-TO-SMN2 | ATGGGATCCATAATTCCCCCACCAC- | 13 |
| 6, 8 | | minigene Nluc- | CTCCCATATGTCCAGATTCTCTT- | |
| | | inactive | GATGATGCTGATGCTTTGGGAAGTATGTTA | |
| | | (exon 6, 8) | ATTTCATGGTACATGAGTGGCTATCAT- | |
| | | | ACTGGCTATTATATGGAAATGCTGGCATA- | |
| | | | GAGCAG- | |
| | | | CACGTCTTCACACTCGAAGATTTCGTTGGG | |
| | | | GACTGGCGACAGACAGCCGGCTACAAC- | |
| | | | CTGGACCAAGTCCTT- | |
| | | | GAACAGGGAGGTGTGTCCAG- | |
| | | | TTTGTTTCAGAATCTCGGGGTGTCCGTAAC | |
| | | | TCCGATCCAAAGGATTGTCCTGAGCGGTGA | |
| | | | AAATGGGCTGAA- | |
| | | | GATCGACATCCATGTCATCATCCCG- | |
| | | | TATGAAGGTCTGAGCGGCGAC- | |
| | | | CAAATGGGCCAGATCGAAAAAATTTTTAA | |
| | | | GGTGGTGTACCCTGTGGATGATCATCAC- | |
| | | | TTTAAGGTGATCCTGCACTATGGCACAC- | |
| | | | TGG- | |
| | | | TAATCGACGGGGTTACGCCGAACATGATC | |
| | | | GACTATTTCGGACGGCCG- | |
| | | | TATGAAGGCATCGCCGTGTTCGACGG- | |
| | | | CAAAAAGATCACTG- | |
| | | | TAACAGGGACCCTGTGGAACGGCAACAAA | |
| | | | ATTATCGAC- | |
| | | | GAGCGCCTGATCAACCCCGAC- | |
| | | | GGCTCCCTGCTGTTCCGAGTAAC- | |
| | | | CATCAAC- | |
| | | | GGAGTGACCGGCTGGCGGCTGTGCGAACG | |
| | | | CATTCTGGCGTAG | |
| SMN2 | NSK163 | CMV-TO-SMN2 | ATGGGATCCATAATTCCCCCACCAC- | 14 |
| 6, 7, 8 | | minigene Nluc- | CTCCCATATGTCCAGATTCTCTT- | |
| | | active | GATGATGCTGATGCTTTGGGAAGTATGTTA | |
| | | (exon 6, 7, 8) | ATTTCATGGTACATGAGTGGCTATCAT- | |
| | | | ACTGGCTATTATATGGGTTTTAGA- | |
| | | | CAAAATCAAAAAGAAGGAAGGTGCTCACA | |
| | | | TTCCTTAAATATAAGGAGAAATGCTGG- | |
| | | | CATAGAGCAGCACGTCTTCACACTCGAA- | |
| | | | GAT- | |
| | | | TTCGTTGGGGACTGGCGACAGACAGCCGG | |
| | | | CTACAACCTGGACCAAGTCCTT- | |
| | | | GAACAGGGAGGTGTGTCCAGTTT- | |
| | | | GTTTCAGAATCTCGGGGTGTCCGTAACTCC | |
| | | | GATCCAAAGGATTGTCCTGAGCGGTGAAA | |
| | | | ATGGGCTGAA- | |
| | | | GATCGACATCCATGTCATCATCCCG- | |
| | | | TATGAAGGTCTGAGCGGCGAC- | |
| | | | CAAATGGGCCAGATCGAAAAAATTTTTAA | |
| | | | GGTGGTGTACCCTGTGGATGATCATCAC- | |
| | | | TTTAAGGTGATCCTGCACTATGGCACAC- | |
| | | | TGG- | |
| | | | TAATCGACGGGGTTACGCCGAACATGATC | |
| | | | GACTATTTCGGACGGCCG- | |
| | | | TATGAAGGCATCGCCGTGTTCGACGG- | |
| | | | CAAAAAGATCACTG- | |
| | | | TAACAGGGACCCTGTGGAACGGCAACAAA | |
| | | | ATTATCGAC- | |
| | | | GAGCGCCTGATCAACCCCGAC- | |
| | | | GGCTCCCTGCTGTTCCGAGTAAC- | |
| | | | CATCAAC- | |
| | | | GGAGTGACCGGCTGGCGGCTGTGCGAACG | |
| | | | CATTCTGGCGTAG | |
| 13 nt | NSK467 | SFFV-mCherry- | AGCATTTCTCCTTA- | 27 |
| | | SGc(Smn2ex7.os | TATTGAAGGAATGCGAGCACCTTGGCAT- | |
| | | 13- | ATGCGAC- | |
| | | 72 bp.lowBG.13 bp | GGCCATGGAAGGTGCTCGCATTTTCATAGC | |
| | | loop)cSG- | CGTCTGTTATGTCCTTCTTTTTGATTTT- | |
| | | EGFP-(3xMS2) | GTCTAAAACCCATATAATA | |
| | | (FLP-IN) | | |
| 16 nt | NSK477 | SFFV-mCherry- | AGCATTTCTCCTTA- | 30 |
| | | SGc(Smn2ex7.os | TATTGAAGGAATGCGAGCACCTTGGCAT- | |
| | | 13- | ATGCGAC- | |
| | | 72 bp.lowBG.16 bp | GGCCATGGAAGGTGCTCGCATTCCTTTCAT | |
| | | loop)cSG- | AGCCGTCTGTTATGTCCTTCTTTTTGATTTT- | |
| | | EGFP-(3xMS2) | GTCTAAAACCCATATAATA | |
| | | (FLP-IN) | | |
| 19 nt | NSK476 | SFFV-mCherry- | AGCATTTCTCCTTA- | 31 |
| | | SGc(Smn2ex7.os | TATTGAAGGAATGCGAGCACCTTGGCAT- | |
| | | 13- | ATGCGAC- | |
| | | 72 bp.lowBG.19 bp | GGCCATGGAAGGTGCTCGCATTCCTTCATT | |
| | | loop)cSG- | CATAGCCGTCTGTTATGTCCTTCTTTTT- | |
| | | EGFP-(3xMS2) | GATTTTGTCTAAAACCCATATAATA | |
| | | (FLP-IN) | | |
| 22 nt | NSK475 | SFFV-mCherry- | AGCATTTCTCCTTA- | 32 |
| | | SGc(Smn2ex7.os | TATTGAAGGAATGCGAGCACCTTGGCAT- | |
| | | 13- | ATGCGAC- | |
| | | 72 bp.lowBG.22 bp | GGCCATGGAAGGTGCTCGCATTCCTTCAAT | |
| | | loop)cSG- | ATTCATAGCCGTCTGTTATGTCCTTCTTTTT | |
| | | EGFP-(3xMS2) | GATTTTGTCTAAAACCCATATAATA | |
| | | (FLP-IN) | | |

### Example 7

### Comparison of Different Length of Low Background NOT Sensor to Original NOT Sensors (FIG. 9)

Cells were transfected with 200 ng plasmid DNA encoding the sensor sequence; 30 ng plasmid DNA encoding ADARp150; and 270 ng plasmid DNA encoding either an unrelated transcript, SCN1A exons 20, 20N, 21, or SCN1A exons 20, 21.

TABLE 6
Sensor nucleotides and target RNAs used in Example 7

| | plasmid | | | SEQ ID |
|---|---|---|---|---|
| | designation | plasmid name | sensor sequence | NO: |
| SCN1a | NSK250 | CMV-TO- | ATGGTTTCATTGGTCAGTTTAACAG- | 22 |
| exons 20, | | SCN1A | CAAATGCCTTGGGTTACTCAGAACTT- | |
| 20N, 21 | | (exons 20, 20N, | GGAGCCATCAAATCTCTCAGGACACTAAGA | |
| | | 21) | GCTCTGAGACCTCTAA- | |
| | | | GAGCCTTATCTCGATTT- | |
| | | | GAAGGGATGAGGGATAATCTT- | |
| | | | GCTCCAACTTGGATGGGGTGGAGCGCTGGT | |
| | | | TCCTCCCCTGAGCCCTTTATTATGGGTGGTT- | |
| | | | GTGAATGCCCTTTTAGGAG- | |
| | | | CAATTCCATCCATCATGAATGTGCTTCTGGT | |
| | | | TTGTCTTATATTCTGGCTAATTTTCAG- | |
| | | | CATCATGGGCGTAAATTTGTTTGCTGG- | |
| | | | CAAATTC- | |
| | | | TACCACTGTATTAACACCACAACTGGTGAC | |
| | | | AGGTTTGACATCGAAGACGTGAATAATCAT- | |
| | | | ACTGATTGCCTAAAACTAATAGAAA- | |
| | | | GAAATGA- | |
| | | | GACTGCTCGATGGAAAAATGTGAAAGTAAA | |
| | | | CTTTGATAATGTAGGATTTGGG- | |
| | | | TATCTCTCTTTGCTTCAAGTTTGTGA | |
| SCN1a | NSK249 | CMV-TO- | ATGGTTTCATTGGTCAGTTTAACAG- | 23 |
| exons 20, | | SCN1A | CAAATGCCTTGGGTTACTCAGAACTT- | |
| 21 | | (exons 20, 21) | GGAGCCATCAAATCTCTCAGGACACTAAGA | |
| | | | GCTCTGAGACCTCTAA- | |
| | | | GAGCCTTATCTCGATTT- | |
| | | | GAAGGGATGAGGGTGGTT- | |
| | | | GTGAATGCCCTTTTAGGAGCAATTCCATCCA | |
| | | | TCATGAATGTGCTTCTGGTTTGTCTTA- | |
| | | | TATTCTGGCTAATTTTCAGCATCATGGGCG- | |
| | | | TAAATTTGTTTGCTGGCAAATTCTACCACTG | |
| | | | TATTAACACCACAACTGGTGACAGGTTT- | |
| | | | GACATCGAAGACGTGAATAATCAT- | |
| | | | ACTGATTGCCTAAAACTAATAGAAAGAAAT | |
| | | | GA- | |
| | | | GACTGCTCGATGGAAAAATGTGAAAGTAAA | |
| | | | CTTTGATAATGTAGGATTTGGG- | |
| | | | TATCTCTCTTTGCTTCAAGTTTGA | |
| 18 nt | NSK464 | SFFV- | ATTCATGATGGATGGAATT- | 33 |
| | | mCherry- | GCTCCTCAAAGGGCATTCACAACCACTGG- | |
| | | SGc(os13.NOT- | CATATGCGAC- | |
| | | SCN1A_20N. | GGCCATGGAGTGGTTGTGAATGCCCTTCCA | |
| | | 45, 64, 45. | TAATAAAGGGCTCAGGGGAGGAAC- | |
| | | lowBG.18 bp | CAGCGCTCCACCCCATCCAAGTTGGAGCAA- | |
| | | loop)cSG- | GAT- | |
| | | EGFP- | TATCTTCATAGCCGTCTGTTATGCCTCATCC | |
| | | (3xMS2) | CTTCAAATCGAGATCAGGCTCTT- | |
| | | (FLP-IN) | GGAGGTCTCAGAGC | |
| 12 nt | NSK465 | SFFV- | ATTCATGATGGATGGAATT- | 34 |
| | | mCherry- | GCTCCTCAAAGGGCATTCACAACCACTGG- | |
| | | SGc(os13.NOT- | CATATGCGAC- | |
| | | SCN1A_20N. | GGCCATGGAGTGGTTGTGAATCCATAATAA | |
| | | 45, 64, 45. | AGGGCTCAGGGGAGGAAC- | |
| | | lowBG.12 bp | CAGCGCTCCACCCCATCCAAGTTGGAGCAA- | |
| | | loop)cSG- | GAT- | |
| | | EGFP- | TATCTTCATAGCCGTCTGTTATGCCTCATCC | |
| | | (3xMS2) | CTTCAAATCGAGATCAGGCTCTT- | |
| | | (FLP-IN) | GGAGGTCTCAGAGC | |
| 6 nt | NSK466 | SFFV- | ATTCATGATGGATGGAATT- | 35 |
| | | mCherry- | GCTCCTCAAAGGGCATTCACAACCACTGG- | |
| | | SGc(os13.NOT- | CATATGCGAC- | |
| | | SCN1A_20N. | GGCCATGGAGTGGTTCCATAATAAAGGGCT | |
| | | 45, 64, 45. | CAGGGGAGGAAC- | |
| | | lowBG.6 bp | CAGCGCTCCACCCCATCCAAGTTGGAGCAA- | |
| | | loop)cSG- | GAT- | |
| | | EGFP- | TATCTTCATAGCCGTCTGTTATGCCTCATCC | |
| | | (3xMS2) | CTTCAAATCGAGATCAGGCTCTT- | |
| | | (FLP-IN) | GGAGGTCTCAGAGC | |
| original | NSK252 | SFFV- | ATTCATGATGGATGGAATT- | 25 |
| NOT | | mCherry- | GCTCCTCAAAGGGCATTCACAACCACTGG- | |
| sensor | | SGc(os13.NOT- | CATATGCGAC- | |
| | | SCN1A_20N. | GGCCATGGACCATAATAAAGGGCTCAGGGG | |
| | | 45, 64, 45)cSG- | AGGAACCAGCGCTCCACCCCATCCAAGTT- | |
| | | EGFP- | GGAGCAAGAT- | |
| | | (3xMS2) | TATCTTCATAGCCGTCTGTTATGCCTCATCC | |
| | | (FLP-IN) | CTTCAAATCGAGATCAGGCTCTT- | |
| | | | GGAGGTCTCAGAGC | |
| 15 nt | NSK481 | SFFV- | ATTCATGATGGATGGAATT- | 36 |
| | | mCherry- | GCTCCTCAAAGGGCATTCACAACCACTGG- | |
| | | SGc(os13.NOT- | CATATGCGAC- | |
| | | SCN1A_20N. | GGCCATGGAGTGGTTGTGAATGCCCCATAA | |
| | | 45, 64, 45. | TAAAGGGCTCAGGGGAGGAAC- | |
| | | lowBG.15 bp | CAGCGCTCCACCCCATCCAAGTTGGAGCAA- | |
| | | loop)cSG- | GAT- | |
| | | EGFP- | TATCTTCATAGCCGTCTGTTATGCCTCATCC | |
| | | (3xMS2) | CTTCAAATCGAGATCAGGCTCTT- | |
| | | (FLP-IN) | GGAGGTCTCAGAGC | |
| 9 nt | NSK482 | SFFV- | ATTCATGATGGATGGAATT- | 37 |
| | | mCherry- | GCTCCTCAAAGGGCATTCACAACCACTGG- | |
| | | SGc(os13.NOT- | CATATGCGAC- | |
| | | SCN1A_20N. | GGCCATGGAGTGGTTGTGCCATAATAAAGG | |
| | | 45, 64, 45. | GCTCAGGGGAGGAAC- | |
| | | lowBG.9 bp | CAGCGCTCCACCCCATCCAAGTTGGAGCAA- | |
| | | loop)cSG- | GAT- | |
| | | EGFP- | TATCTTCATAGCCGTCTGTTATGCCTCATCC | |
| | | (3xMS2) | CTTCAAATCGAGATCAGGCTCTT- | |
| | | (FLP-IN) | GGAGGTCTCAGAGC | |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112 (f) or 35 U.S.C. § 112 (6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112 (6) is not invoked.

Notwithstanding the appended claims, the disclosure set forth herein is also described by the following clauses:

1. A method of screening a candidate agent for activity in altering a first target RNA in a cell, the method comprising:
a) contacting the candidate agent with the cell comprising a sensor RNA, wherein the sensor RNA comprises:
   (i) a first nucleotide sequence comprising a sensor nucleotide sequence that hybridizes to the target RNA, wherein the sensor nucleotide sequence comprises one or more editable codons,
   (ii) a second nucleotide sequence encoding a first cleavage domain, and
   (iii) a third nucleotide sequence encoding an output protein; and
b) assaying for the presence of the output protein to assess the activity of the candidate agent to alter the target RNA.

2. The method of clause 1, wherein the first nucleotide sequence comprising the sensor nucleotide sequence comprises:
(a) a first region that is reverse complementary to the first target RNA and a second target RNA,
(b) a second region comprising a stem-loop sequence comprising one or more editable codons wherein a first portion of the stem-loop sequence is reverse complementary to the second target RNA, and
(c) a third region that is reverse complementary to the first target RNA and the second target RNA

3. The method of clause 2, wherein the first target RNA and the second target RNA are different splice variants of the same gene.

4. The method of any of clauses 1-3, wherein the first target RNA is a pathogenic splice variant.

5. The method of clauses 2 or 3, wherein the second target RNA is a pathogenic splice variant.

6. The method of clauses 2 or 3, wherein the first target RNA and the second target RNA are non-pathogenic splice variants.

7. The method of any one of clauses 3-6, wherein the gene is selected from the group consisting of: ABCA4, ABCB4, ABCD1, ACADSB, ADA, ADAMTS13, AGL, ALB, ALAS, ALDH3A2, ALG6, APC, APOB, APP, AR, ATM, ATP7A, ATR, B2M, BCL6, BMP2K, BRCA1, BRCA2, BTK, C3, CAT, CD46, CDH1, CDH23, CFTR, CHM, COL11A1, COL11A2, COL1A1, COL1A2, COL2A1, COL3A1, COL4A5, COL6A1, COL6A3, COL7A1, COL9A2, COLQ, CUL4B, CYBB, CYP17, CYP19, CYP27, CYP27A1, DES, DMD, DYSF, EGFR, EMD, ESR1, ETV4, F13A1, F5, F7, F8, FAH, FANCA, FANCC, FANCG, FBN1, FECH, FGA, FGFR2, FGG, FIX, FLNA, FOXM1, FRAS1, GALC, GH1, GHV, HADHA, HAO1, HBA2, HBB, HEXA, HEXB, HLCS, HMBS, HMGCL, HNFIA, HPRT1, HPRT2, HSF4, HSPG2, HTT, IDS, IKBKAP, INSR, ITGB2, ITGB3, JAG1, KRAS, KRT5, KRAS, LICAM, LAMA3, LDLR, LMNA, LPL, LRRK2, MADD, MAPT, MeCP2, MLH1, MSH2, MSTIR, MTHFR, MUT, MVK, MYC, NF1, NF2, OAT, OPA1, OTC, PAH, PBGD, PCCA, PCSK9, PDH1, PGK1, PHEX, PKD2, PKLR, PLEKHM1, PLKR, POMT2, PRDM1, PRKAR1A, PROC, PSEN1, PTCH1, PTEN, PYGM, RP6KA3, RPGR, RSK2, SBCAD, SCN1A, SCN5A, SERPINA1, SERPINC1, SLC12A3, SLC26A4, SLC6A8, SMN2, SPINK5, SPTA1, SYNGAP1, TP53, TRAPPC2, TSC1, TSC2, TSHB, TTR, UGT1A1, and USH2A.

8. The method of any one of clauses 2-7, wherein the first target RNA is an RNA comprising exons 6 and 8 of SMN2 and the second target RNA is an RNA comprising exons 6, 7, and 8 of SMN2.

9. The method of clause 8, wherein the first region is reverse complementary to exon 8 of SMN2, the third region is reverse complementary to exon 6 of SMN2, and the first portion of the stem-loop sequence is reverse complementary to exon 7 of SMN2.

10. The method of clause 9, wherein the third region is reverse complementary to exon 8 of SMN2, the first region is reverse complementary to exon 6 of SMN2, and the first portion of the stem-loop sequence is reverse complementary to exon 7 of SMN2.

11. The method of any one of clauses 2-7, wherein the first target RNA is an RNA comprising exons 20 and 21 of SCN1A and the second target RNA is an RNA comprising exons 20, 20N, and 21 of SCN1A.

12. The method of clause 11, wherein the first region is reverse complementary to exon 21 of SCN1A, the third region is reverse complementary to exon 20 of SCN1A, and the first portion of the stem-loop sequence is reverse complementary to exon 20N of SCN1A.

13. The method of clause 11, wherein the third region is reverse complementary to exon 21 of SCN1A, the first region is reverse complementary to exon 20 of SCN1A, and the first portion of the stem-loop sequence is reverse complementary to exon 20N of SCN1A.

14. The method of any one of clauses 2-13, wherein the first region is 5′ of the second region and the second region is 5′ of the third region.

15. The method of any one of clauses 2-13, wherein the editable codon comprises one or more bases that are mismatched with a sequence within the stem-loop opposite the one or more editable codons.

16. The method of any one of clauses 2-15, wherein a second portion of the stem-loop sequence is reverse complementary to the first region or the third region.

17. The method of clause 16, wherein the second portion of the stem-loop sequence is reverse complementary to the first region.

18. The method of clause 17, wherein the second portion of the stem-loop sequence is reverse complementary to the third region.

19. The method of any one of clauses 2-18, wherein the stem-loop sequence comprises a stem-sequence and a loop sequence.

20. The method of clause 19, wherein the first portion of the stem-loop sequence is contained in the loop sequence of the stem-loop sequence.

21. The method of one of clauses 19 or 20, wherein the second portion of the stem-loop sequence is contained in the loop sequence of the stem-loop sequence.

22. The method of any one of clauses 19-21, wherein the loop sequence of the stem-loop sequence is 10-31 nucleotides in length.

23. The method of any one of clauses 19-22, wherein the first portion of the stem-loop sequence is 1/10 to ¾ the total length of the loop sequence of the stem-loop sequence.

24. The method of any one of clauses 19-23, wherein the second portion of the loop sequence of the stem-loop sequence is 1/10 to ¾ the total length of the loop sequence of the stem-loop sequence.

25. The method of any one of clauses 1-24, wherein the activity in altering the first target RNA results in the first target RNA being alternatively spliced.

26. The method of any one of clauses 1-25, wherein the activity in altering the first target RNA results in a reduction in the expression of the first target RNA.

27. The method of any one of clauses 2-26, wherein the activity in altering the first target RNA results in an increase in the expression of the first target RNA.

28. The method of any one of clauses 2-27, wherein the activity in altering the first target RNA results in a change in the nucleic acid sequence of the first target RNA.

29. The method of any one of clauses 2-28, wherein the first target RNA is associated with a disease or a condition.

30. The method of any one of clauses 2-29, wherein the first target RNA is mRNA.

31. The method of any one of clauses 2-30, wherein the first target RNA is a non-coding RNA.

32. The method of clause 31, wherein the non-coding RNA is selected from the group consisting of microRNA, long non-coding RNA, ribosomal RNA, transfer RNA, small interfering RNA, piwi-interacting RNA, small nucleolar RNA, and small nuclear RNA.

33. The method of any one of clauses 1-32, wherein the cell is a tissue-specific cell.

34. The method of any one of clauses 1-33, wherein the cell is a disease-related cell.

35. The method of any one of clauses 1-34, wherein the cell is a human patient-derived cell.

36. The method of any one of clauses 1-35, wherein the cell is present in an organoid.

37. The method of any one of clauses 1-36, wherein the cell is present in a non-human animal.

38. The method of any one of clauses 1-37, wherein the candidate agent is selected from the group consisting of a nucleic acid, a small molecule, a polypeptide, and an antibody or fragment thereof.

39. The method of clause 38, wherein the candidate agent is a nucleic acid.

40. The method of clause 39, wherein the nucleic acid is an antisense oligonucleotide.

41. The method of clause 40, wherein the nucleic acid is a small interfering RNA.

42. The method of clause 38, wherein the candidate agent is a small molecule.

43. The method of any one of clauses 1-42, wherein the editable codon is a stop codon, a start codon or an AUA codon.

44. The method of any one of clauses 1-43, wherein the cleavage domain is a 2A self-cleaving domain is selected from the group of T2A, P2A, E2A, and F2A.

45. The method of any one of clauses 1-44, wherein the output protein is selected from the group consisting of: a fluorescent protein, a luminescent protein, a transcription factor, a tag, and a recombinase.

46. The method of any one of clauses 1-45, wherein the assaying comprises using microscopy, flow cytometry, a plate reader, immunoblotting, or a combination thereof.

47. The method of any one of clauses 1-46, wherein a presence of the output protein is employed as an indication that the candidate has activity in altering the target RNA in the cell.

48. The method of any one of clauses 1-47, wherein an absence of the output protein is employed as an indication that the candidate has activity in altering the target RNA in the cell.

49. The method of any one of clauses 1-48, wherein a change in the amount of the output protein is employed as an indication that the candidate has activity in altering the target RNA in the cell.

50. The method of clause 49, wherein the change in the amount of the output protein is a decrease in the amount of the output protein.

51. The method of clause 49, wherein the change in the amount of the output protein is an increase in the amount of the output protein.

52. The method of any one of clauses 1-51, further comprising:
(i) a fourth nucleotide sequence comprising a second cleavage domain wherein the fourth nucleotide sequence precedes the first nucleotide sequence, and
(ii) a fifth nucleotide sequence comprising a nucleotide sequence encoding a marker protein wherein the fifth nucleotide sequence precedes the fourth nucleotide sequence.

53. The method of any one of clauses 1-52, further comprising contacting the cell with an adenosine deaminase acting on RNA (ADAR) protein or a coding sequence thereof.

54. The method of clause 53, wherein the ADAR protein is selected from the group consisting of ADAR2, ADARP110, ADARP150, a modified ADAR comprising an ADAR deaminase domain and an RNA motif binding domain, and a modified ADAR comprising a nuclear localization signal.

55. The method of clause 8, wherein the sensor nucleotide sequence comprises a sequence that is at least 70% identical to a sequence selected from the group consisting of: SEQ ID NO: 20, SEQ ID NO: 27, SEQ ID NO: 28, SEQ ID NO: 29, SEQ ID NO: 30, SEQ ID NO: 31, and SEQ ID NO: 32.

56. The method of clause 8, wherein the sensor nucleotide sequence comprises a sequence that is at least 80% identical to a sequence selected from the group consisting of: SEQ ID NO: 20, SEQ ID NO: 27, SEQ ID NO: 28, SEQ ID NO: 29, SEQ ID NO: 30, SEQ ID NO: 31, and SEQ ID NO: 32.

57. The method of clause 8, wherein the sensor nucleotide sequence comprises a sequence that is at least 90% identical to a sequence selected from the group consisting of: SEQ ID NO: 20, SEQ ID NO: 27, SEQ ID NO: 28, SEQ ID NO: 29, SEQ ID NO: 30, SEQ ID NO: 31, and SEQ ID NO: 32.

58. The method of clause 11, wherein the sensor nucleotide sequence comprises a sequence that is at least 70% identical to a sequence selected from the group consisting of: : SEQ ID NO: 24, SEQ ID NO: 25, SEQ ID NO: 26, SEQ ID NO: 33, SEQ ID NO: 34, SEQ ID NO: 35, SEQ ID NO: 36, and SEQ ID NO: 37.

59. The method of clause 11, wherein the sensor nucleotide sequence comprises a sequence that is at least 80% identical to a sequence selected from the group consisting of: : SEQ ID NO: 24, SEQ ID NO: 25, SEQ ID NO: 26, SEQ ID NO: 33, SEQ ID NO: 34, SEQ ID NO: 35, SEQ ID NO: 36, and SEQ ID NO: 37.

60. The method of clause 11, wherein the sensor nucleotide sequence comprises a sequence that is at least 90% identical to a sequence selected from the group consisting of: : SEQ ID NO: 24, SEQ ID NO: 25, SEQ ID NO: 26, SEQ ID NO: 33, SEQ ID NO: 34, SEQ ID NO: 35, SEQ ID NO: 36, and SEQ ID NO: 37.

61. The method of clause 1, wherein the sensor nucleotide sequence comprises a sequence that is at least 70% identical to a sequence selected from the group consisting of: SEQ ID NO: 40, SEQ ID NO: 41, and SEQ ID NO: 42.

62. The method of clause 1, wherein the sensor nucleotide sequence comprises a sequence that is at least 80% identical to a sequence selected from the group consisting of: SEQ ID NO: 40, SEQ ID NO: 41, and SEQ ID NO: 42.

63. The method of clause 1, wherein the sensor nucleotide sequence comprises a sequence that is at least 90% identical to a sequence selected from the group consisting of: SEQ ID NO: 40, SEQ ID NO: 41, and SEQ ID NO: 42.

64. A nucleic acid comprising any one of the sensor RNA of any one of the preceding clauses.

65. A recombinant vector comprising the sensor RNA of any one of the preceding clauses.

66. The recombinant vector of clause 65, wherein the recombinant vector is selected from the group of a plasmid, a viral vector, a cosmid, and an artificial chromosomc.

67. A kit comprising:
the cell of any one of clauses 1-66.

68. The kit of clause 67, further comprising an adenosine deaminase acting on RNA (ADAR) protein or a coding sequence thereof.

69. The kit of clause 67, wherein the ADAR protein is selected from the group consisting of ADAR2, ADARP110, ADARP150, a modified ADAR comprising an ADAR deaminase domain and an RNA motif binding domain.

70. The kit of any one of clauses 67-69, further comprising a candidate agent that has activity in altering the target RNA in the cell and a candidate agent that does not have activity in altering the target RNA in the cell.

What is claimed is:

1. A method of screening a candidate agent for activity in altering a first target RNA in a cell, the method comprising:
a) contacting the candidate agent with the cell comprising a sensor RNA, wherein the sensor RNA comprises:
(i) a first nucleotide sequence comprising a sensor nucleotide sequence that hybridizes to the target RNA, wherein the sensor nucleotide sequence comprises one or more editable codons,
(ii) a second nucleotide sequence encoding a first cleavage domain, and
(iii) a third nucleotide sequence encoding an output protein; and
b) assaying for the presence of the output protein to assess the activity of the candidate agent to alter the target RNA.

2. The method of claim 1, wherein the first nucleotide sequence comprising the sensor nucleotide sequence comprises:
(a) a first region that is reverse complementary to the first target RNA and a second target RNA,
(b) a second region comprising a stem-loop sequence comprising one or more editable codons wherein a first portion of the stem-loop sequence is reverse complementary to the second target RNA, and
(c) a third region that is reverse complementary to the first target RNA and the second target RNA

3. The method of claim 2, wherein the first target RNA and the second target RNA are different splice variants of the same gene.

4. The method of any one of claims 1-3, wherein the first target RNA or the second target RNA is a pathogenic splice variant.

5. The method of claim 2 or 3, wherein the first target RNA and/or the second target RNA are non-pathogenic splice variants.

6. The method of any one of claims 3-5, wherein the gene is selected from the group consisting of: ABCA4, ABCB4, ABCD1, ACADSB, ADA, ADAMTS13, AGL, ALB, ALAS, ALDH3A2, ALG6, APC, APOB, APP, AR, ATM, ATP7A, ATR, B2M, BCL6, BMP2K, BRCA1, BRCA2, BTK, C3, CAT, CD46, CDH1, CDH23, CFTR, CHM, COL11A1, COL11A2, COL1A1, COL1A2, COL2A1, COL3A1, COL4A5, COL6A1, COL6A3, COL7A1, COL9A2, COLQ, CUL4B, CYBB, CYP17, CYP19, CYP27, CYP27A1, DES, DMD, DYSF, EGFR, EMD, ESR1, ETV4, F13A1, F5, F7, F8, FAH, FANCA, FANCC, FANCG, FBN1, FECH, FGA, FGFR2, FGG, FIX, FLNA, FOXM1, FRAS1, GALC, GH1, GHV, HADHA, HAO1, HBA2, HBB, HEXA, HEXB, HLCS, HMBS, HMGCL, HNF1A, HPRT1, HPRT2, HSF4, HSPG2, HTT, IDS, IKBKAP, INSR, ITGB2, ITGB3, JAG1, KRAS, KRT5, KRAS, LICAM, LAMA3, LDLR, LMNA, LPL, LRRK2, MADD, MAPT, MeCP2, MLH1, MSH2, MSTIR, MTHFR, MUT, MVK, MYC, NF1, NF2, OAT, OPA1, OTC, PAH, PBGD, PCCA, PCSK9, PDH1, PGK1, PHEX, PKD2, PKLR, PLEKHM1, PLKR, POMT2, PRDM1, PRKAR1A, PROC, PSEN1, PTCH1, PTEN, PYGM, RP6KA3, RPGR, RSK2, SBCAD, SCN1A, SCN5A, SERPINA1, SERPINC1, SLC12A3, SLC26A4, SLC6A8, SMN2, SPINK5, SPTA1, SYNGAP1, TP53, TRAPPC2, TSC1, TSC2, TSHB, TTR, UGT1A1, and USH2A.

7. The method of any one of claims 2-6, wherein the first target RNA is an RNA comprising exons 6 and 8 of SMN2 and the second target RNA is an RNA comprising exons 6, 7, and 8 of SMN2.

8. The method of claim 7, wherein the first region is reverse complementary to exon 8 of SMN2, the third region is reverse complementary to exon 6 of SMN2, and the first portion of the stem-loop sequence is reverse complementary to exon 7 of SMN2.

9. The method of any one of claims 2-6, wherein the first target RNA is an RNA comprising exons 20 and 21 of SCN1A and the second target RNA is an RNA comprising exons 20, 20N, and 21 of SCN1A.

10. The method of claim 9, wherein the first region is reverse complementary to exon 21 of SCN1A, the third region is reverse complementary to exon 20 of SCN1A, and the first portion of the stem-loop sequence is reverse complementary to exon 20N of SCN1A.

11. The method of any one of claims 2-10, wherein a second portion of the stem-loop sequence is reverse complementary to the first region or the third region.

12. The method of any one of claims 2-11, wherein the stem-loop sequence comprises a stem-sequence and a loop sequence.

13. The method of claim 12, wherein the first portion and the second portion of the stem-loop sequence is contained in the loop sequence of the stem-loop sequence.

14. The method of any one of claims 1-13, wherein the activity in altering the first target RNA results in:
a) the first target RNA being alternatively spliced,
b) a reduction in the expression of the first target RNA,
c) an increase in the expression of the first target RNA, or
d) a change in the nucleic acid sequence of the first target RNA.

15. The method of any one of claims 1-14, wherein the cell is a cell selected from the group consisting of: a tissue-specific cell, a disease-related cell, a human patient-derived cell, a cell present in an organoid, and a cell present in a non-human animal.

16. The method of any one of claims 1-15, wherein the candidate agent is selected from the group consisting of a nucleic acid, a small molecule, a polypeptide, and an antibody or fragment thereof.

17. The method of any one of claims 1-16, wherein the output protein is selected from the group consisting of: a fluorescent protein, a luminescent protein, a transcription factor, a tag, and a recombinase.

18. The method of any one of claims 1-17, wherein the assaying comprises using microscopy, flow cytometry, a plate reader, immunoblotting, or a combination thereof.

19. The method of any one of claims 1-18, wherein:
a) a presence of the output protein is employed as an indication that the candidate has activity in altering the target RNA in the cell,
b) an absence of the output protein is employed as an indication that the candidate has activity in altering the target RNA in the cell, or
c) a change in the amount of the output protein is employed as an indication that the candidate has activity in altering the target RNA in the cell.

20. The method of any of claims 1-19, further comprising:
(i) a fourth nucleotide sequence comprising a second cleavage domain wherein the fourth nucleotide sequence precedes the first nucleotide sequence, and
(ii) a fifth nucleotide sequence comprising a nucleotide sequence encoding a marker protein wherein the fifth nucleotide sequence precedes the fourth nucleotide sequence.


SEQUENCE LISTING (42 sequences)

SEQ ID NO: 1 - RNA, 63 nt, synthetic construct
  source 1..63: mol_type = other RNA
caaggtcaatgaggagatgtacatagaaatacaatcctgtgtacatcttctagcatgacccac

SEQ ID NO: 2 - RNA, 57 nt, synthetic construct
  source 1..57: mol_type = other RNA
caaggtcaatgaggagatgtacataatacaatgtgtacatcttctagcatgacccac

SEQ ID NO: 3 - RNA, 90 nt, synthetic construct
  source 1..90: mol_type = other RNA
cccaacctctgtctactcaccacagccccccagcatcactgtgaatgctgccatggatgctagagggctacaggaagagccagaagttgg

SEQ ID NO: 4 - RNA, 60 nt, synthetic construct
  source 1..60: mol_type = other RNA
ctcaccacagccccccagcatcactgtgaatgctgccatggatgctagagggctacagga

SEQ ID NO: 5 - RNA, 51 nt, synthetic construct
  source 1..51: mol_type = other RNA
aagtggcatatgcgacggccatggactggttcatagccgtctgttatgcct

SEQ ID NO: 6 - RNA, 45 nt, synthetic construct
  source 1..45: mol_type = other RNA
tggcatatgcgacggccatggactggttcatagccgtctgttatg

SEQ ID NO: 7 - RNA, 48 nt, synthetic construct
  source 1..48: mol_type = other RNA
cattaaggtgggtggaatagtatacaaagtatcccacctaccctgatg

SEQ ID NO: 8 - RNA, 48 nt, synthetic construct
  source 1..48: mol_type = other RNA
cattaaggtgggtggaatagtatacaaagtatcccacctaccccgatg

SEQ ID NO: 9 - RNA, 51 nt, synthetic construct
  source 1..51: mol_type = other RNA
tccgtttaggtgggtggaatagtaatacaaagtatcccacctacccagacg

SEQ ID NO: 10 - RNA, 35 nt, synthetic construct
  source 1..35: mol_type = other RNA
catttaggtgggtgggctaaccacctacccagatg

SEQ ID NO: 11 - RNA, 41 nt, synthetic construct
  source 1..41: mol_type = other RNA
catttaggtgggtggaatgctaaatcccacctacccagatg

SEQ ID NO: 12 - RNA, 36 nt, synthetic construct
  source 1..36: mol_type = other RNA
atgcgacggccatggactggttcatagccgtctgtt

SEQ ID NO: 13 - RNA, 656 nt, synthetic construct
  source 1..656: mol_type = other RNA
atgggatccataattcccccaccacctcccatatgtccagattctcttgatgatgctgatgctttgggaagtatgttaatttcatggtacatgagtggctatcatactggctattatatggaaatgctggcatagagcagcacgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtag

SEQ ID NO: 14 - RNA, 711 nt, synthetic construct
  source 1..711: mol_type = other RNA
atgggatccataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaatataaggagaaatgctggcatagagcagcacgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtag

SEQ ID NO: 15 - RNA, 117 nt, synthetic construct
  source 1..117: mol_type = other RNA
agcatttctccttatattgaaggaatgcgagcaccttggcatatgcgacggccatggactggttcatagccgtctgttatgtccttctttttgattttgtctaaaacccatataata

SEQ ID NO: 16 - RNA, 72 nt, synthetic construct
  source 1..72: mol_type = other RNA
cttatatttaaggaatgtgagcaccttccttctttttagttttgtctaaaacccatatcatggccagtatgg

SEQ ID NO: 17 - RNA, 72 nt, synthetic construct
  source 1..72: mol_type = other RNA
agcatttctccttatatttcaggaatgtgggcaccttagttctttttgattttgtctaaaacccatataata

SEQ ID NO: 18 - RNA, 72 nt, synthetic construct
  source 1..72: mol_type = other RNA
cagcatttctccttatatttaaggaatgtgagcacctagcttctttttgattttgtctcaaacccatataat

SEQ ID NO: 19 - RNA, 72 nt, synthetic construct
  source 1..72: mol_type = other RNA
tatgccagcatttctccttatatttcaggaatgtaggcaccttccttctttttgattttgtctaaaacccat

SEQ ID NO: 20 - RNA, 187 nt, synthetic construct
  source 1..187: mol_type = other RNA
cgaaatcttcgagtgtggagacgtgctgctctatgccagcatttctggcatatgcgacggccatggatccttatatttaaggaatgtgagcaccttccttctttttggttttgtctaaaaccttcatagccgtctgttatgcatataatagccagtatgatagccactcatgtaccatggaattcaa

SEQ ID NO: 21 - RNA, 168 nt, synthetic construct
  source 1..168: mol_type = other RNA
cgagtgtggagacgtgctgctctatgccagcatttctggcatatgcgacggccatggatccttatatttaaggaatgtgagcaccttccttctttttggttttgtctaaaaccttcatagccgtctgttatgcatataatagccagtatgatagccactcatgtacca

SEQ ID NO: 22 - RNA, 477 nt, synthetic construct
  source 1..477: mol_type = other RNA
atggtttcattggtcagtttaacagcaaatgccttgggttactcagaacttggagccatcaaatctctcaggacactaagagctctgagacctctaagagccttatctcgatttgaagggatgagggataatcttgctccaacttggatggggtggagcgctggttcctcccctgagccctttattatgggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagtttgtga

SEQ ID NO: 23 - RNA, 411 nt, synthetic construct
  source 1..411: mol_type = other RNA
atggtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagtttga

SEQ ID NO: 24 - RNA, 207 nt, synthetic construct
  source 1..207: mol_type = other RNA
aagcacattcatgatggatggaattgctcctcaaagggcattcacaaccactggcatatgcgacggccatggaccataataaagggctcaggggaggaaccagcgctccaccccatccaagttggagcaagattatcttcatagccgtctgttatgcctcatcccttcaaatcgagatcaggctcttggaggtctcagagctcttgg

SEQ ID NO: 25 - RNA, 195 nt, synthetic construct
  source 1..195: mol_type = other RNA
attcatgatggatggaattgctcctcaaagggcattcacaaccactggcatatgcgacggccatggaccataataaagggctcaggggaggaaccagcgctccaccccatccaagttggagcaagattatcttcatagccgtctgttatgcctcatcccttcaaatcgagatcaggctcttggaggtctcagagc

SEQ ID NO: 26 - RNA, 120 nt, synthetic construct
  source 1..120: mol_type = other RNA
agcatttctccttatattgaaggaatgcgagcaccttggcatatgcgacggccatggaaggtgctttcatagccgtctgttatgtccttctttttgattttgtctaaaacccatataata

SEQ ID NO: 27 - RNA, 126 nt, synthetic construct
  source 1..126: mol_type = other RNA
agcatttctccttatattgaaggaatgcgagcaccttggcatatgcgacggccatggaaggtgctcgcattttcatagccgtctgttatgtccttctttttgattttgtctaaaacccatataata

SEQ ID NO: 28 - RNA, 123 nt, synthetic construct
  source 1..123: mol_type = other RNA
agcatttctccttatattgaaggaatgcgagcaccttggcatatgcgacggccatggaaggtgctcgcttcatagccgtctgttatgtccttctttttgattttgtctaaaacccatataata

SEQ ID NO: 29 - RNA, 129 nt, synthetic construct
  source 1..129: mol_type = other RNA
agcatttctccttatattgaaggaatgcgagcaccttggcatatgcgacggccatggaaggtgctcgcattcctttcatagccgtctgttatgtccttctttttgattttgtctaaaacccatataata

SEQ ID NO: 30 - RNA, 132 nt, synthetic construct
  source 1..132: mol_type = other RNA
agcatttctccttatattgaaggaatgcgagcaccttggcatatgcgacggccatggaaggtgctcgcattccttcattcatagccgtctgttatgtccttctttttgattttgtctaaaacccatataata

SEQ ID NO: 31 - RNA, 135 nt, synthetic construct
  source 1..135: mol_type = other RNA
agcatttctccttatattgaaggaatgcgagcaccttggcatatgcgacggccatggaaggtgctcgcattccttcaatattcatagccgtctgttatgtccttctttttgattttgtctaaaacccatataata

SEQ ID NO: 32 - RNA, 213 nt, synthetic construct
  source 1..213: mol_type = other RNA
attcatgatggatggaattgctcctcaaagggcattcacaaccactggcatatgcgacggccatggagtggttgtgaatgcccttccataataaagggctcaggggaggaaccagcgctccaccccatccaagttggagcaagattatcttcatagccgtctgttatgcctcatcccttcaaatcgagatcaggctcttggaggtctcagagc

SEQ ID NO: 33 - RNA, 207 nt, synthetic construct
  source 1..207: mol_type = other RNA
attcatgatggatggaattgctcctcaaagggcattcacaaccactggcatatgcgacggccatggagtggttgtgaatccataataaagggctcaggggaggaaccagcgctccaccccatccaagttggagcaagattatcttcatagccgtctgttatgcctcatcccttcaaatcgagatcaggctcttggaggtctcagagc

SEQ ID NO: 34 - RNA, 201 nt, synthetic construct
  source 1..201: mol_type = other RNA
attcatgatggatggaattgctcctcaaagggcattcacaaccactggcatatgcgacggccatggagtggttccataataaagggctcaggggaggaaccagcgctccaccccatccaagttggagcaagattatcttcatagccgtctgttatgcctcatcccttcaaatcgagatcaggctcttggaggtctcagagc

SEQ ID NO: 35 - RNA, 210 nt, synthetic construct
  source 1..210: mol_type = other RNA
attcatgatggatggaattgctcctcaaagggcattcacaaccactggcatatgcgacggccatggagtggttgtgaatgccccataataaagggctcaggggaggaaccagcgctccaccccatccaagttggagcaagattatcttcatagccgtctgttatgcctcatcccttcaaatcgagatcaggctcttggaggtctcagagc

SEQ ID NO: 36 - RNA, 204 nt, synthetic construct
  source 1..204: mol_type = other RNA
attcatgatggatggaattgctcctcaaagggcattcacaaccactggcatatgcgacggccatggagtggttgtgccataataaagggctcaggggaggaaccagcgctccaccccatccaagttggagcaagattatcttcatagccgtctgttatgcctcatcccttcaaatcgagatcaggctcttggaggtctcagagc

SEQ ID NO: 37 - RNA, 143 nt, synthetic construct
  source 1..143: mol_type = other RNA
atgggatccataattcccccaccacctcccatatgtccagattctcttgatgatgctgatgctttgggaagtatgttaatttcatggtacatgagtggctatcatactggctattatatggaaatgctggcatagagcagcac

SEQ ID NO: 38 - RNA, 198 nt, synthetic construct
  source 1..198: mol_type = other RNA
atgggatccataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaatataaggagaaatgctggcatagagcagcac

SEQ ID NO: 39 - RNA, 117 nt, synthetic construct; identical to SEQ ID NO: 15
  source 1..117: mol_type = other RNA

SEQ ID NO: 40 - RNA, 135 nt, synthetic construct
  source 1..135: mol_type = other RNA
ctctatgccagcatttctccttatattgaaggaatgcgagcaccttggcatatgcgacggccatggactggttcatagccgtctgttatgtccttctttttgattttgtctaaaacccatataatagccagtatg

SEQ ID NO: 41 - RNA, 147 nt, synthetic construct
  source 1..147: mol_type = other RNA
gtgctgctctatgccagcatttctccttatattgaaggaatgcgagcaccttggcatatgcgacggccatggactggttcatagccgtctgttatgtccttctttttgattttgtctaaaacccatataatagccagtatgatagcc

SEQ ID NO: 42 - RNA, 147 nt, synthetic construct; identical to SEQ ID NO: 41
  source 1..147: mol_type = other RNA